(19) United States
(12) Patent Application Publication
(10) Pub. No.: US 2014/0080906 A1
(43) Pub. Date: Mar. 20, 2014

(54) FAT EMULSION PROVIDING TASTE MASKING FOR ACTIVE HEALTH AND WELLNESS INGREDIENTS

(71) Applicant: Antioxidant Superfoods, Inc., Naples, FL (US)

(72) Inventors: Gary ERVIN, Naples, FL (US); Erin ERVIN, Naples, FL (US)

(73) Assignee: Antioxidant Superfoods, Inc., Naples, FL (US)

(21) Appl. No.: 13/840,071

(22) Filed: Mar. 15, 2013

Related U.S. Application Data
(63) Continuation-in-part of application No. 13/223,794, filed on Sep. 1, 2011.
(60) Provisional application No. 61/378,939, filed on Sep. 1, 2010.
(60) Provisional application No. 61/474,553, filed on Apr. 12, 2011.

(51) Int. Cl. A61K 9/107; A61K 31/225; A23D 9/007

(52) U.S. Cl. CPC A61K 9/107; A23D 9/007; A61K 31/225
USPC 514/547

(57) ABSTRACT

The embodiments relate to fat emulsion structures based on both an aqueous and non-aqueous glycerin component as the primary aqueous component in which the fat emulsion can create a wide range of viscosities that mimic fat structures similar to cream, or all the way to hardened fat structures like Trans Fat. The fat emulsion can be added to a wide group of foods that use a monosaccharide or disaccharide as the basis for its sweetener component, can lower the sugar content of foods, can improve mouth feel while lowering the fat content in high fat foods, can add a balance of dietary fats and fiber to foods, and can add antioxidant content to food products. The fat emulsion also can be used as a taste masking composition to mask the taste of unpalatable active ingredients or unpalatable components that may be added to the emulsion.

### RELATED APPLICATIONS

This application is a continuation-in-part of U.S. patent application Ser. No. 13/223,794, entitled: “A Fat Emulsion Providing Improved Health and Taste Characteristics in Food,” filed on Sep. 1, 2011, currently pending, which claims priority to provisional patent application Ser. No. 61/378,939 filed Sep. 1, 2010, and to provisional patent application Ser. No. 61/474,553, filed Apr. 12, 2011, the disclosures of each of which are incorporated by reference herein in their entireties.

### BACKGROUND

1. Field of the Invention

The embodiments relate to fat emulsions structures based on both an aqueous and non-aqueous glycerin component as the primary aqueous component. A wide range of viscosities can be made that can mask the taste and deliver therapeutic dosing levels of over-the-counter, pharmaceutical, nutraceutical or medicine active ingredients. These fat emulsions can be made into many common delivery methods for actives in the health and wellness category including; gummies, chews, mixable powders, liquid dosing, etc. thus improving consumer compliance and dramatically reduce consumer discomfort.

2. Description of Related Art

Edible fats currently available in the marketplace come in several different formats. First, fats are available as sole fats like palm oil, palm kernel oil, coconut oil, cocoa butter, shea butter, butter fat, milk fat, etc. These types of fats are usually used within confections and in foods that need a harder fat texture or a rich and creamy taste. Chocolate, ice cream, cookies, baked goods, confections like caramel, yogurt, cottage cheese, etc are examples of foods containing these types of fats. Other foods contain only softer edible fats like canola oil, soybean oil, sunflower oil, safflower oil, olive oil, nut oils, etc. are used in foods that need softer less viscous fats like salad dressings, mayonnaise, humus, etc. Some foods use both a harder fat and a softer fat called inter-esterfied within their composition like nutrition bars, snack foods, baked goods like cookies, confections, etc. Food categories that use these softer fats that have monounsaturated and polyunsaturated oils have some health benefits. Edible oils are a necessary part of a healthy diet but the ratio of edible oils should be more of the healthier monounsaturated and polyunsaturated oils and less of the saturated fat oils especially animal based saturated fat oils like milk fat.

Second, many foods use fats that are partially and fully hydrogenated within their compositions either alone or in combination with a softer oil like canola or soybean oil. These oils have been hydrogenated and become harder than the original oils, but are cheaper than the high saturated fat oils like palm kernel oil or coconut oil. Examples of partially or fully hydrogenated oils are partially hydrogenated cottonseed oil, partially hydrogenated soybean oil, fully hydrogenated coconut oil or fully hydrogenated palm oil. During the hydrogenation process Trans Fatty Acids are produced as bi-products in the chemical adjustment of the oils. These Trans Fatty Acids are proven to have negative health ramifications and the below website reference by the American Heart Association explains that ¾ of all the Trans Fatty Acids in Americans diets come from these hydrogenated oils. http://www.americanheart.org/presenter.jhtml?identifier=532

Many snack foods use partially hydrogenated oils including cookies, baked goods, cakes, granola bars, nutrition bars, coffee creamers, pudding, cake icing, cake mixes, ice cream confections, confections including top brands like Snickers, Milkyway, Twix, Butterfinger, Baby Ruth, Hershey's Cookies and Cream, Rollos, Starbursts, Skittles, Airheads, Caramel filled Chocolates and many other types of confections and food categories. Partially and fully hydrogenated oils have become the leading added fat after cocoa butter in the chocolate confection and overall confection industry. Foods that are made with certain vegetable shortening have hydrogenated fats within them. Shortening is one of the most common ingredients in baked goods. These hydrogenated oils that include Trans Fat have been directly linked to coronary heart disease.

Third, foods use Trans Fat within their composition. Categories of foods like pre-made cookies, cookies, baked goods, frying oil made from Trans Fat, etc. add this synthetic fat into our diets. Trans Fat was created originally to replace the “bad for you” saturated fats in the diet. Global marketers moved to add Trans Fat into their foods over the last several decades because of the texture and mouth feel that the Trans Fat produces in foods that it produces. Over the last decade the scientific community realized after extensive research that Trans Fat causes the largest risk for negative health problems like cardiovascular disease and heart disease compared to other fats available to be added into foods. Trans Fat became the largest fat problem within the US and global food systems. Companies started removing Trans Fat out of their foods and reformulating them. The problem is that the solution to replacing Trans Fat was to use fully hydrogenated or partially hydrogenated oils in place of Trans Fat to create similar texture and mouth feel in the products. Science today shows that these hydrogenated oils add the same Trans Fat to foods that the Trans Fat they replaced do, just in smaller amounts. Today food manufacturers and marketers are looking for innovative ways to create fats that can provide the rheological properties of Trans Fat without the negative health ramifications.

In some instances, a thickened fat is desired to provide dimensional stability (e.g., to minimize fluid flow or keep a fat ingredient in a particular location in a food article). At present, only three techniques for providing a thickened fat such as for use in a food product are practical. The first, hydrogenation, as described above, while useful and widely practiced undesirably results in the generation of Trans fatty acids. The second technique involves addition of silicon dioxide to oil. For example, U.S. Pat. No. 3,669,681 describes preparing a shortening for products which are heated prior to consumption, wherein edible oils are mixed with silicon dioxide and a bridging compound with the result being a shortening that is asserted to not weep or run at elevated temperatures while retaining desirable mouth feel characteristics of the untreated oil. While useful, current food regulatory restrictions restrict employment of this technique. The third technique involves addition of sufficient amounts of hardstock, whether naturally occurring (such as palm oil hardstock) or synthetic (such as produced by hydrogenation), to an oil to provide desired amounts of thickening or hardening. Again, while useful, health concerns favor minimization of consumption of such hardstock ingredients.

U.S. Pat. No. 7,736,684 issued Jun. 15, 2010 to Plank, et. al describes the preparation of viscous fat compositions with low amounts of Trans Fat. This patent describes a fat composition with less than 40% Trans Fat that has cyclodextrin bound to the fat and it claims the final fat composition can be substantially more viscous or harder based on the invention.

While creating fat compositions that have lower amounts of Trans Fat like Plank, et. al describes within its invention would be an improvement over using 100% Trans Fat in foods the fat composition still delivers Trans Fat that has proven negative health ramifications including causing cardiovascular and heart disease.

U.S. Pat. No. 6,649,207 issued Nov. 18, 2003 to Coote, et al. describes the preparation of a an oil in water emulsion that essentially has zero water consisting of a fat or fat blend, a cold hydrating viscous agent that include one of the following; cold swelling starch, inulin and gums and optional protein. This final oil in water fat composition is made by mixing the ingredients at ambient temperatures. Coote, et al. suggests that the goal of its invention is to produce a final emulsion that is water continuous compared to the goal of our invention that is to create a final emulsion that is fat continuous. Coote, et al. suggests that Inulin could be used within its invention, but the preferable ingredient is a starch for its invention. We found through experimentation that if our ingredients were mixed at ambient temperatures that a viscous fat composition was also formed, but it took heating the composition to 150 and mixing it vigorously until the composition thickened into a more viscous harder fat composition. Once this composition is cooled it turns into a solid continuous fat phase composition. Increasing the amount of dietary fiber in our composition can make the final continuous fat phase almost solid after the cooling process occurs.

U.S. Pat. No. 6,663,910 to Soumya, et al., describes the preparation of food products with carrageen that have a carbohydrate sweetener component and a gelling component. This patent is said to facilitate a high solids level while being pourable at a higher temperature. This product also has moisture content at 13% to 25%. These products differ from our invention in that we have several edible oil components and a dietary fiber component within in our invention. The products of Soumya, et al., are essentially a flavored chewy or gummy candy confection with a completely different structure.

The link below discusses the Harvard School of Public Health study that was the largest food study ever conducted to examine how certain fats affect consumer's long term health. The conclusion is that consumers need to get more of the “good unsaturated fats” (unsaturated fats including monounsaturated and polyunsaturated fats) and less of the saturated fats in their diets. http://www.hsph.harvard.edu/nutritionsoure/what-should-you-eat/fats-full story/index.html

This report is based on the largest study ever undertaken on the effect of reducing saturated fats and replacing them with unsaturated fats including monounsaturated and polyunsaturated fats. The study concluded that many of the major health issues facing consumers today can be directly attributed to consumption of high levels of saturated fat, partially hydrogenated, fully hydrogenated, and Trans fats. The study examined the effect of replacing such saturated fats with healthier monounsaturated and polyunsaturated fats in the diet and how certain saturated fats like those found in coconut are significantly better for you than saturated fat found in milk and butter. The study concluded that this had a significant effect on reducing the risks of developing chronic health conditions including cardiovascular disease and coronary heart disease. Categories of foods that are high in saturated fat and where embodiments of the invention can be used to improve the health characteristics and reduce the fat content include, but are not limited to ice cream, milk, cheese, yogurt, butter, cream cheese, salad dressings, vegetable dips, granola, fried foods, confections including chocolate and many other food categories.

Many poor tasting actives, wellness ingredients, etc., currently are delivered in formats that attempt to improve the consumer convenience. Some examples of poor tasting actives that have been utilized with more convenient delivery vehicles in the health and wellness industry include; Amoxicillin sold in a flavored liquid, Metamucil's Smooth Textured Psyllium Powder sold in a mixable powder, and Docusate Sodium sold in a flavored liquid. These examples of poor tasting actives are sold in these delivery methods, but they still have an undesirable taste. These unpalatable tasting actives typically are provided in tablets, gel caps, capsules, or flavored liquids because of their undesirable taste which makes more convenient delivery methods unfeasible. This lack of convenience in delivery methods for poor tasting active ingredients leads to lack of compliance in taking these actives and consumer discomfort.

Some industry references show that Smooth Textured Psyllium powder still only has 26% compliance by the consumer because of the poor taste. Other actives like dioctyl sodium sulfosuccinate or docusate sodium, a stool softener, are incorporated primarily in tablets, capsules or flavored unit dosing products that are all inconvenient and the current liquid delivery methods for docusate sodium are still unpalatable, but the consumer has no better option to take the active. The compliance of these actives is low due to the inconvenience of taking an unpleasant tasting product or the lack of convenient forms of taking the actives. Many other actives in the health and wellness category have such poor tastes that to date no more palatable delivery methods other than tablets or capsules have been created.

Taste masking of undesirable pharmaceuticals often is a laborious process, and must take into consideration the particular taste receptors affected, and how the taste can be effectively masked. Ayenew, et al., “Trends in Pharmaceutical Taste Masking Technologies: A Patent Review,” Recent Patent on Drug Delivery & Formulation 2009, vol. 3, pp. 26-39 (2009) discloses a variety of different techniques used to mask the undesirable taste of different active ingredients. For example, hydrophobic polymers have been used to coat bitter medications, such as famotidine, to assist in delivery. Other coatings have been used to deliver pivoxil sulbactam, ranitidine, clarithromycin, cetirizine dihydrochloride, and the like. Other taste masking techniques include granulation, use of sweeteners, microencapsulation, (liposomes) taste suppressants and potentiators, solid dispersions, ion exchange resins, viscosity enhancers, and formation of complexes of the active with other chemicals, such as cyclodextrin. Other taste masking techniques are disclosed in Deepak, et al., “Taste Masking Technologies: . . . ,” Int. Research J. of Pharmacy, 2012, 2(4), pp. 108-116 (2012).

There exists a need to develop formulations capable of masking the undesirable taste of actives and wellness ingredients, especially in the geriatric, pediatric, and animal fields. Embodiments described herein are capable of creating desirable tasting liquids, chews, or powders based on a fat emulsion technology that completely masks poor tasting actives.

The description herein of certain advantages and/or disadvantages of known products or methods are not intended to remove some or all of the components of these known products or methods from the scope of the present embodiments. Indeed, some embodiments described herein may incorporate some or all of their components of the known products or methods, without suffering from their inherent disadvantages. The description provided in this section also is not intended as an admission that the documents or products are prior art, especially the description of the embodiments described herein.

### SUMMARY

A feature of an embodiment is its ability to mask unpleasant tasting health and wellness actives through a fat emulsion's ability to agglomerate the actives and be combined with flavors, sweeteners, and food acids that further mask their taste. Another embodiment permits a wide range of fat emulsions to be formulated that can deliver low fat contents down to 0.3% fat, and high contents of dietary fiber up to 70% while having the physical and organoleptic characteristics of higher fat emulsions or fat. The fat emulsions are superior to known fat emulsions with respect to the ability to agglomerate and mask unpalatable actives, provide functional benefits of delivering dietary fiber and healthier unsaturated fats and its ability to be solid at room temperature so that a powder can be produced.

Another feature of an embodiment of the invention is to provide well balanced nutritional structures using the swelling properties of dietary fibers, and potentially other hydrocolloids, as well as corn starch to stabilize the water activity and harden the composition so that hard stock oils that normally would be needed to create a hard composition at room temperature are needed in lower levels or aren't needed at all, when compared to known fat emulsion compositions.

Dietary fiber has specific benefits to consumer's health as well as to the skin especially helping to support hydration. Dietary fiber also has benefits to consumers including its ability to combine with other ingredients to create a hardened composition. Another feature of an embodiment is the combination of ingredients that together create a synergistic composition that masks unpalatable actives delivered in the health and wellness category. Unpalatable active ingredients such as docusate sodium, glucosamine and chondroitin, amoxicillin, prenatal vitamins, etc. can be delivered in a palatable fat emulsion that in certain embodiments can be a fluid liquid, a soft or hard chew, or a powder that can be dissolved by stirring it into a liquid such as water, milk, juice, etc. Additional embodiments permit the fat emulsion to be used to make a wide range of functional foods that can deliver therapeutic levels of actives. Examples of some composition forms include, but are not limited to, gummies, chews, granola bars, cereal bars, cereal clusters, baked goods like brownies, cookies, muffins, snack bars, or any other unit dosing format of food that can make use of the fat emulsion described herein to deliver therapeutic levels of specific actives sold within health and wellness whether it be a pharmaceutical, nutraceutical, herb, over the counter medicine, a medicine or any other ingredient that claims to have certain benefits for health and wellness.

In accordance with these and other features of embodiments, there is provided a fat emulsion comprising: (a) from about 0.3% to about 40% by weight of the composition of an edible oil; (b) from about 0.001% to about 70% by weight of the composition of a dietary fiber component; (c) from about 1% to about 40% by weight of the composition of a water component or glycerin component; (d) from about 0.001% to about 70% by weight of the composition of a thickening component; and (e) from about 0.001% to about 50% of a pharmaceutical active ingredient or health wellness ingredient. The composition also may contain from about 0.003% to about 40% of a flavorant in the form of, for example, an extract, from about 0.001% to about 25% of a sugar alcohol component, from about 0.001% to about 15% of an organic acid component, and from about 0.001% to about 10% of an emulsifier component. The composition also may optionally contain, in some embodiments, one or more additives selected from the group consisting of an additional flavor component, a color component, a preservative component, a stabilizing component, a gelling component such as gelatin, gellan gum, xanthan gum, pectin, carrageenan, cellulose gum, gum arabic, modified starch and combinations thereof, a supplement component such as citric acid, malic acid, lactic acid, fumaric acid, tartaric acid and adipic acid, a nutraceutical component, a vitamin, a mineral, a medicine component, or combinations thereof.

In accordance with these and other features of embodiments, there is provided a fat emulsion comprising: (a) from about 0.3% to about 80% by weight of the composition of an edible oil; (b) from about 0.001% to about 70% by weight of the composition of a dietary fiber component; (c) from about 1% to about 60% by weight of the composition of a water component or glycerin component; and (d) from about 0.001% to about 35% by weight of the composition of a thickening component. The composition also may contain from about 0.001% to about 10% of an emulsifier component, from 0% to 15% of a glycerin component (in the event water is used as component (c)), up to 5% of a hydrocolloid structure, up to 25% of a protein component, and up to 5% of an acidulant. The composition also may optionally contain, in some embodiments, one or more additives selected from the group consisting of a flavor component, a color component, a preservative component, a stabilizing component, a gelling component such as gelatin, gellan gum, xanthan gum, pectin, carrageenan, cellulose gum, gum arabic, modified starch and combinations thereof, a supplement component such as citric acid, malic acid, lactic acid, fumaric acid, tartaric acid and adipic acid, a nutraceutical component, a vitamin, a mineral, a medicine component, or combinations thereof.

In accordance with another feature of an embodiment of the invention, there is provided a method of making a taste masking homogeneous fat emulsion comprising: (a) mixing from about 0.3% to about 40% by weight of an edible oil, from about 1% to about 40% by weight of water or glycerin, and from about 0.003% to about 40% of a flavorant in the form of, for example, an extract in a suitable mixing vessel capable of mixing and heating; (b) mixing the components and heating the mixing vessel to a temperature within the range of from about 100° F. to about 150° F., preferably within the range of from about 110° F. to about 130° F., and most preferably about 120° F.; (c) adding from about 0.001% to about 70% by weight of a dietary fiber component, and heating to a temperature within the range of from about 120° F. to about 160° F., preferably within the range of from about 130° F. to about 150° F., and most preferably about 140° F., for a sufficient period of time to adequately mix the dietary fiber; (d) adding from about 0.001% to about 70% by weight of the composition of a thickening component, and mixing for a period of time to adequately mix and dissolve the thickening component, while maintaining the temperature within the range of from about 120° F. to about 160° F., preferably within the range of from about 130° F. to about 150° F., and most preferably about 140° F.; (e) adding from about 0.001% to about 50% of a pharmaceutical active ingredient or health wellness ingredient, optionally after cooling if the pharmaceutical active ingredient or health wellness ingredient is sensitive to temperature and might otherwise degrade at a given temperature; and (f) cooling the mixture.

The embodiments also encompass adding additional components to the mixture either before, during, or after heating. In a preferred embodiment, the method includes adding from about 0.001% to about 25% of a sugar alcohol component, from about 0.001% to about 15% of an organic acid component, and from about 0.001% to about 10% of an emulsifier component prior to cooling, and raising the temperature of the mixture to a temperature within the range of from about 140° F. to about 170° F., preferably within the range of from about 145° F. to about 160° F., and most preferably about 150° F., for a period of time to adequately dissolve the sugar alcohol component and the organic food acid component. From about 0.001% to about 50% of a pharmaceutical active ingredient or health wellness ingredient can be added at a lower temperature based on the temperature sensitivity of the active ingredient. In the case of temperature sensitive active ingredients they can be added into the mixture as it cools down to make sure the active ingredient does not have any deterioration based on temperature. The temperature of the emulsion can be lowered to 85° F. or less to have these types of temperature sensitive actives added to the composition. The mixture may, if the pharmaceutical active ingredient added is not temperature sensitive, have its temperature increased to 150° F. to 160° F., and mixed under shear for 5 to more than 30 minutes until it thickens into a homogeneous fat emulsion. The temperature then preferably can be lowered while mixing in additional components, with agitation.

The method provides a unique fat emulsion that is capable of masking unpleasant tasting active ingredients, and can harden into a solid at room temperature that can be grinded into a fine flowable powder. The product also could be cryogenically milled in the liquid stage before the emulsion cools to produce a flowable powder, or it can be sparyed onto a carrying agent and then milled into a powder. An example of a carrying agent includes cyclodextrin, maltodextrin and other carrying agents known in the art of milling powders. Anti-caking agents may also be used to improve the flowable characteristics of the final fat emulsion that has now been turned into a powder. Additional sugar alcohol sweeteners and additional powerful natural or artificial flavors can also be added at the milling stage of the fat emulsion.

In accordance with another feature of an embodiment of the invention, there is provided a method of making a homogeneous fat emulsion comprising: (a) mixing from about 0.3% to about 80% by weight of an edible oil, from about 0.001% to about 70% by weight of a dietary fiber component, from about 1% to about 60% by weight of water or glycerin, and from about 0.001% to about 35% by weight of a thickener in a suitable mixing vessel capable of high shear mixing and heating; (b) mixing the components under high shear and heating the mixing vessel to a temperature within the range of from about 140° F. to about 170° F., preferably within the range of from about 150° F. to about 160° F.; and (c) cooling the mixture. The embodiments also encompass adding additional components to the mixture either before or after heating. In a preferred embodiment, the method includes first adding the water or glycerin component, then adding the dietary fiber (and/or corn starch component), followed by the edible oil/fat component and optional emulsifier component. The mixture would then preferably have its temperature increased to 150° F. to 160° F., and mixed under shear for 5 to 30 plus minutes until it thickens into a homogeneous fat emulsion structure. The temperature then preferably can be lowered while mixing in additional components, with agitation.

These and other features and advantages of the embodiments will become readily apparent to those skilled in the art upon review of the Detailed Description that follows.

### DETAILED DESCRIPTION OF PREFERRED EMBODIMENTS

All percentages and ratios are calculated by weight unless otherwise indicated. All percentages and ratios are calculated based on the total final composition unless otherwise indicated.

Referenced herein may be trade names for components including various ingredients utilized in the present invention. The inventors herein do not intend to be limited by materials under a given trade name. Equivalent materials (e.g., those obtained from a different source under a different name or reference number) to those referenced by trade name may be substituted and utilized in the description.

As used herein “fiber” or “dietary fiber” generally denotes a material derived from plant cell walls and which is not digestible by human digestive enzymes, including soluble fiber and insoluble fiber. The fiber component can be naturally derived or synthetic. A portion of the fiber component can be non-starch polysaccharides, including soluble and non soluble fiber. As used herein “soluble fiber” denotes, for example and without limitation, plant gums and oligosaccharides, or modified gums, modified celluloses, non-starch polysaccharides that are soluble in water, some of which can form viscous gels.

The embodiments provide a fat emulsion that is capable of masking the undesirable taste of certain unpalatable active ingredients. Those skilled in the art will appreciate, however, that the fat emulsion described herein can be used with any pharmaceutical active or wellness component, or other unpalatable components that are intended to be ingested by, for example, humans and animals (pets, zoo animals, etc.), and is not limited to pharmaceutical actives or wellness ingredients. When used to mask the undesirable taste of an unpalatable component, or when used to carry a pharmaceutical agent or wellness ingredient, the fat emulsion preferably comprises: (a) from about 0.3% to about 40% by weight of the composition of an edible oil; (b) from about 0.001% to about 70% by weight of the composition of a dietary fiber component; (c) from about 1% to about 40% by weight of the composition of a water component or glycerin component; (d) from about 0.001% to about 70% by weight of the composition of a thickening component; and (e) from about 0.001% to about 50% of a pharmaceutical active ingredient or health wellness ingredient. The composition also may contain from about 0.003% to about 40% of a flavorant in the form of, for example, an extract, from about 0.001% to about 25% of a sugar alcohol component, from about 0.001% to about 15% of an organic acid component, and from about 0.001% to about 10% of an emulsifier component. The composition also may optionally contain, in some embodiments, one or more additives selected from the group consisting of an additional flavor component, a color component, a preservative component, a stabilizing component, a gelling component such as gelatin, gellan gum, xanthan gum, pectin, carrageenan, cellulose gum, gum arabic, modified starch and combinations thereof, a supplement component such as citric acid, malic acid, lactic acid, fumaric acid, tartaric acid and adipic acid, a nutraceutical component, a vitamin, a mineral, a medicine component, or combinations thereof.

A fat emulsion that masks unpalatable actives delivered in the health and wellness category might comprise: (a) from about 0.3% to about 40% an edible oil component, more specifically 2% to 50% of an edible oil component, even more specifically 3% to 25% of an edible oil component, and most specifically 4% to 15% of an edible oil component, by weight of the composition; (b) from about 0.001% to about 70% a dietary fiber component, more specifically 2% to 60% a dietary fiber component, even more specifically 5% to 50% a dietary fiber component, and most specifically 10% to 40% a dietary fiber component, by weight of the composition; (c) from about 1% to about 40% a water component or glycerin component, more specifically 2% to 40% a water component or glycerin component, even more specifically 3% to 20% a water component or glycerin component, and most specifically 4% to 10% a water component or glycerin component, by weight of the composition; (d) from about 0.001% to about 70% a thickening component, more specifically 5% to 60% a thickening component, and most specifically 10% to 35% a thickening component, by weight of the composition; (e) from about 0.001% to about 25% a strawberry extract component, more specifically 1% to 15% a strawberry extract component, even more specifically 3% to 10% a strawberry extract component, and most specifically 5% to 8% a strawberry extract component, by weight of the composition; (f) from about 0.001% to about 25% a lemon extract component, more specifically 1% to 15% a lemon extract component, even more specifically 3% to 10% a lemon extract component, and most specifically 4% to 8% a lemon extract component, by weight of the composition; (g) from about 0.001% to 25% liquid vanilla extract, more specifically 1% to 15% a vanilla extract component, even more specifically 3% to 10% a vanilla extract component, and most specifically 3.5% to 8% a vanilla extract component, by weight of the composition; (h) from about 0.001% to 25% a sugar alcohol component, more specifically 0.25% to 15% a sugar alcohol component, even more specifically 0.5% to 10% a sugar alcohol component, and most specifically a 1% to 8% sugar alcohol component, by weight of the composition; (i) from about 0.001% to 25% an organic acid component, more specifically 1% to 15% an organic acid component, even more specifically 2% to 10% an organic acid component, and most specifically a 3% to 8% organic acid component, by weight of the composition; (j) from about 0.001% to about 10% of an emulsifier component, more specifically a 0.5% to 8% emulsifier component, even more specifically a 0.75% to 5% emulsifier component, and most specifically a 1% to 3% emulsifier component, by weight of the composition; and (m) from about 0.001% to 50% an active ingredient from the category of pharmaceutical, nutraceutical, over the counter drugs and prescription medicines, more specifically 0.5% to 40% an active ingredient from the category of pharmaceutical, nutraceutical, over the counter drugs and prescription medicines, even more specifically 0.75% to 30% an active ingredient from the category of pharmaceutical, nutraceutical, over the counter drugs and prescription medicines, and most specifically 1% to 20% an active ingredient from the category of pharmaceutical, nutraceutical, over the counter drugs and prescription medicines, by weight of the composition.

The embodiments also provide a fat emulsion comprising: (a) from about 0.3% to about 80% by weight of the composition of an edible oil; (b) from about 0.001% to about 70% by weight of the composition of a dietary fiber component; (c) from about 1% to about 60% by weight of the composition of a water component or glycerin component; and (d) from about 0.001% to about 35% by weight of the composition of a thickening component. The composition also may contain from about 0.001% to about 10% of an emulsifier component, from 0% to 15% of a glycerin component (in the event water is used as component (c)), up to 5% of a hydrocolloid structure, up to 25% of a protein component, and up to 5% of an acidulant. The composition also may optionally contain, in some embodiments, one or more additives selected from the group consisting of a flavor component, a color component, a preservative component, a stabilizing component, a gelling component like gelatin or pectin, a supplement component, a nutraceutical component, a medicine component or combinations thereof.

Other embodiments include a method of making a taste masking fat emulsion comprising: (a) mixing from about 0.3% to about 40% by weight of an edible oil, from about 1% to about 40% by weight of water or glycerin, and from about 0.003% to about 40% of a flavorant in the form of, for example, an extract in a suitable mixing vessel capable of mixing and heating; (b) mixing the components and heating the mixing vessel to a temperature within the range of from about 100° F. to about 150° F., preferably within the range of from about 110° F. to about 130° F., and most preferably about 120° F.; (c) adding from about 0.001% to about 70% by weight of a dietary fiber component, and heating to a temperature within the range of from about 120° F. to about 160° F., preferably within the range of from about 130° F. to about 150° F., and most preferably about 140° F., for a sufficient period of time to adequately mix the dietary fiber; (d) adding from about 0.001% to about 70% by weight of the composition of a thickening component, and mixing for a period of time to adequately mix and dissolve the thickening component, while maintaining the temperature within the range of from about 120° F. to about 160° F., preferably within the range of from about 130° F. to about 150° F., and most preferably about 140° F.; (e) adding from about 0.001% to about 50% of a pharmaceutical active ingredient or health wellness ingredient, optionally after cooling if the pharmaceutical active ingredient or health wellness ingredient is sensitive to temperature and might otherwise degrade at a given temperature; and (f) cooling the mixture.

The embodiments also encompass adding additional components to the mixture either before, during, or after heating. In a preferred embodiment, the method includes adding from about 0.001% to about 25% of a sugar alcohol component, from about 0.001% to about 15% of an organic acid component, and from about 0.001% to about 10% of an emulsifier component prior to cooling, and raising the temperature of the mixture to a temperature within the range of from about 140° F. to about 170° F., preferably within the range of from about 145° F. to about 160° F., and most preferably about 150° F., for a period of time to adequately dissolve the sugar alcohol component and the organic food acid component. From about 0.001% to about 50% of a pharmaceutical active ingredient or health wellness ingredient can be added at a lower temperature based on the temperature sensitivity of the active ingredient. In the case of temperature sensitive active ingredients they can be added into the mixture as it cools down to make sure the active ingredient does not have any deterioration based on temperature. The temperature of the emulsion can be lowered to 85° F. or less to have these types of temperature sensitive actives added to the composition. The mixture may, if the pharmaceutical active ingredient added is not temperature sensitive, have its temperature increased to 150° F. to 160° F., and mixed under shear for 5 to more than 30 minutes until it thickens into a homogeneous fat emulsion. The temperature then preferably can be lowered while mixing in additional components, with agitation.

The method provides a unique fat emulsion that is capable of masking unpleasant tasting active ingredients, and can harden into a solid at room temperature that can be grinded into a fine flowable powder. The product also could be cryogenically milled in the liquid stage before the emulsion cools to produce a flowable powder, or it can be sparyed onto a carrying agent and then milled into a powder. An example of a carrying agent includes cyclodextrin, maltodextrin and other carrying agents known in the art of milling powders. Anti-caking agents may also be used to improve the flowable characteristics of the final fat emulsion that has now been turned into a powder. Additional sugar alcohol sweeteners and additional powerful natural or artificial flavors can also be added at the milling stage of the fat emulsion.

The embodiments also provide a method of making a homogeneous fat emulsion comprising: (a) mixing from about 0.3% to about 80% by weight of an edible oil, from about 0.001% to about 70% by weight of a dietary fiber component, from about 1% to about 60% by weight of water or glycerin, and from about 0.001% to about 35% by weight of a thickener in a suitable mixing vessel capable of high shear mixing and heating; (b) mixing the components under high shear and heating the mixing vessel to a temperature within the range of from about 140° F. to about 170° F., preferably within the range of from about 150° F. to about 160° F.; and (c) cooling the mixture.

In another preferred embodiment of the invention fat emulsion structures can be created that have low levels of fat, on the order of from 0.3% to 10% by weight that have the physical and organoleptic characteristics of higher fat emulsions or fats. The unique ability of the inventive fat emulsion to incorporate dietary fiber, preferably Inulin and/or corn starch, and in some cases a hydrocolloid like cellulose to stabilize the water activity, creates these low fat structure emulsions that mimic high fat emulsions or fats themselves. The addition of Inulin at levels as high as 70% of the structure delivers beneficial dietary fiber to the inventive fat emulsion structure, when compared to known oil/fat emulsion systems. When used to mask the undesirable taste of an unpalatable component, the addition of high levels of thickeners, such as corn starch, also increase the ability of the emulsion to swell and create a relatively hard composition that can be processed into tablets, chews, powders, and the like.

Another embodiment of the invention is that fat emulsions can be made using only liquid at room temperature fats that have predominate levels of unsaturated fats like, for example, canola oil, sunflower oil, soybean oil, olive oil, etc. Typical fat emulsions need to have a hard stock oil/fat or hard at room temperature as part of the fat structure in order to make more viscous fat emulsions. The present embodiments provide for viscous fat emulsions to be created by the swelling of the Inulin and/or corn starch component, and some cases additional hydrocolloid like cellulose, to act as the hard stock oil/fat within the fat emulsion structure. The embodiments are capable of employing 40% to higher oils like canola oil within the fat emulsion structures and the finished emulsions will be viscous just as the case would be if a hard stock as in typical fat emulsions were used. Accordingly, the hard stock oil/fat or hard at room temperature oils/fats used in conventional fat emulsions can be replaced and/or entirely removed using the embodiments of the present invention.

In other embodiments, a soluble dietary fiber component consisting of psyllium husk agglomerated with maltodextrin and sprayed with citric acid or psyllium husk milled into fine powder to improve its solubility can be used as part of, or as the entire dietary fiber component. The ability to agglomerate psyllium husks and improve the taste thereof also permits the use of psyllium husk as part of, or the entire dietary fiber component. The use of a psyllium husk agglomerated with maltodexrin and sprayed with citric acid or psyllium husk milled into fine powder allows certain fat emulsions created within this invention to allow foods to make an FDA Heart Healthy Claim based on soluble fiber. In addition, the ability to agglomerate and mask the taste of psyllium husk agglomerated with maltodextrin and sprayed with citric acid or psyllium husk milled into fine powder allows certain fat emulsions created to deliver therapeutic levels of psyllium husk in a mixable powder form. An average serving of the food application must have 1.7 g of Psyllium Husk within its structure. The food must also be low fat and have 3 grams or less total fat, 1 g or less saturated fat, and low cholesterol or 10% or less daily value or 30 milligrams or less. An important physical attribute of the inventive fat emulsion products is that they can create full fat taste, texture, mouth feel and functional characteristics in a food system while adding Psyllium Husk at therapeutic levels sufficient to satisfy the FDA fat content requirements. This allows fat emulsions to be made within the present invention that, when incorporated in a wide range of food products, can allow the FDA Heart Healthy Soluble Fiber nutritional claim to be made which must deliver 1.7 g of Psyllium Husk per equivalent serving size of a food to make the qualified health claim.

In other embodiments glycerin can be added, acidulants, or protein or combinations of all three can be utilized to improve the stability of the fat emulsion products. Different stability and water activity levels are typically desired for different food and skin care applications and this can be adjusted through the addition of these types of fat emulsion stabilizing ingredients. Glycerin can be used in certain embodiments of this invention as the primary solvent, and it has low moisture content around 6% and can have moisture content as low as 0.3%. When the fat emulsion is used to formulate a taste masking composition, it is preferred that glycerin be used. The preferred embodiments also utilize Inulin and/or starch as well as optional hydrocolloids like cellulose within its structure to completely stabilize moisture content/water activity within the glycerin solvent to create fat emulsions with very low fat contents and low water activity down to 0.3 or lower that allows for many applications that aqueous based fat emulsions can't be utilized within. This includes creating fillings for cookie centers, fillings for donuts, spraying applications for adding spices and flavors to baked snacks, crackers, and chips, chocolate, chocolate coatings, compound coatings, peanut butter and other food applications that must have a low water activity for functionality. The invention contemplates the use of dietary fibers other than inulin and/or corn starch.

In other preferred embodiments, the fat emulsions can be created for frying applications that don't require hard stock oil at high levels as do typical fat emulsions used for frying applications. The hard stock is typically needed to create the correct viscosity of the fat/oil phase within the fat emulsion for frying. The present invention can utilize a majority of liquid oils at room temperature and only a small percentage of hardened oil to none at all as part of the fat phase within the invention. Hard stock fat/oils typically have high levels of saturated fats, hydrogenated fats or Trans Fats within them and contribute to negative health ramifications, when used for frying. The advantage of this hardened or hard stock like fat structure created within the inventive fat emulsion is that it contributes low levels of fat/oil content to the product, and is a feature of the invention's ability to create very low fat emulsions that can be used for frying and which don't create spatter problems.

Another preferred embodiment of the fat emulsion is that the nutritional components necessary to create a complete nutritional food can be incorporated into its structure. A feature of water, dietary fiber, starch, essential fatty acids, protein, minerals and vitamins can be incorporated into a great tasting fat emulsion structure. The levels of the components can be controlled in the inventive fat emulsion such that the exact nutrient balance of the following components can be met:
Proteins—essential to growth and repair of muscle and other body tissues
Fats—one source of energy and important in relation to fat soluble vitamins
Carbohydrates—our main source of energy
Minerals—those inorganic elements occurring in the body and which are critical to its normal functions
Vitamins—water and fat soluble vitamins play important roles in many chemical processes in the body
Water—essential to normal body function—as a vehicle for carrying other nutrients and because 60% of the human body is water
Roughage—the fibrous indigestible portion of our diet essential to health of the digestive system

The exact proportions of each of the above nutrients can create an ideal nutritional food and the fat emulsion structure can accomplish this which should include the following:

57% Carbohydrates (sugar, sweets, bread, cakes)

30% Fats (dairy products, oil)

13% Protein (eggs, milk, meat, poultry, fish)

25 to 35 grams dietary fiber daily for roughage

Water sufficient for proper hydration

Minerals, vitamins, and nutrients necessary for sustaining health

The embodiments described herein can provide a good taste, texture, melt, and mouth feel fat emulsion composition without the necessity of utilizing high levels of saturated fat, partially or fully hydrogenated fats, or Trans Fats that all have negative health consequences. This improved health fat emulsion composition can be an addition to current foods or it can replace the use of the fats that make up certain foods. The fat emulsion compositions of the embodiments can have an improved balance of dietary fats including saturated fats, polyunsaturated fats, and monounsaturated fats including beneficial Omega 6 and Omega 3 fatty acids. The embodiments also have an antioxidant flavor profile that gives the fat emulsion composition a specific unique flavor that preferably stems from the synergistic use of coconut oil, vanilla powder, and the edible oils described herein. The flavor profile thus created is a completely unique taste profile vs. other fat compositions within the market place.

The fat emulsion composition preferably has a beneficial dietary fiber component. This dietary fiber component along with the edible oils and water component forms a fat emulsion composition with varying levels of viscosity from a pourable fat composition to a solid Trans Fat like fat composition. Within certain embodiments, the fat compositions can have the same or similar viscosity as partially hydrogenated and fully hydrogenated oils and the viscosity of shortening. The dietary fiber component, specifically Inulin, also helps create the texture and mouth feel of a true fat as well as positive health attributes. Dietary fiber can be included so that the final product using the fat composition can claim to have up to 35% Daily Value Dietary Fiber per serving and up to 50% Daily Value Dietary Fiber per serving in certain food categories. Dietary Fiber has been proven to help reduce the risk of colon cancer, improve digestion health, balance blood sugar, and has some probiotic benefits.

The embodiments described herein also are capable of reducing the sugar content in foods by 25% to over 50%. The preferred embodiments can lower the sugar content by replacing it with healthy edible oils, an antioxidant flavor profile blend, beneficial dietary fiber, water or glycerin, and additional potential supplement ingredients like calcium in certain compositions. Eating foods high in sugar is tied directly to negative health issues including; being overweight, obesity, type II diabetes, cardiovascular disease, heart disease and other health issues. Many foods have sugar contents in the 30% to over 50% sugar level and Americans and global consumers are eating high sugar foods at an alarming rate. Many of these high sugar foods also use partially or fully hydrogenated oils within them that also add to the negative health characteristics of these foods. The fat emulsion can lower the sugar content and replace the partially hydrogenated and fully hydrogenated or even Trans Fat within foods while maintaining a similar taste, texture, and mouth feel of the product. The preferred use of Inulin as one of the primary ingredients within the fat emulsion composition adds some sweetness to the composition while adding the positive mouth feel and texture of a fat. While not intending on being bound by any theory of operation, Inulin is believed to create a synergistic effect of the ingredients that can reduce the sugar content in foods while replacing the necessity for poor health fats like the partially hydrogenated oils, fully hydrogenated oils and Trans Fat within the foods.

Glycerin based fat emulsions created within the present invention have a sweet taste and also can reduce sugar while maintaining excellent taste within food applications. In addition, glycerin based fat emulsions can be particularly useful in formulating taste masking compositions. Chocolate, chocolate confections and chewy confections especially have high sugar contents and certain glycerin based fat emulsions made within this invention can lower sugar contents while adding mouth feel similar to partially, fully hydrogenated and palm oil or other high saturated fats.

Another aspect of the embodiments helps to reduce the calorie content in foods through the use of the fat emulsion compositions. The dietary fiber component is capable of replacing other high calorie ingredients commonly used in confections and other fat-containing compositions, and consequently, use of the fat emulsions enables the reduction of the calorie content in various food products, including flavored chewy candy confection. Sugar has a calorie content of around 4.5 calories per gram and fat has a calorie content of around 8.5 calories per gram compared to Inulin, fructoligiosaccharides, and other forms of dietary fiber, which only have 2 calories per gram or lower.

In certain preferred embodiments, fructooligiosaccharides can be used as the dietary fiber source and this will lower the calorie content of the composition and thus lower the content of the food using the fat emulsion composition. The use of fructooligiosaccharides and water or glycerin can reduce calories by 20% to 30%, when compared to regular formulas with similar taste profiles to the original food. Calories in many foods that have high sugar contents and that have fat or high fat content are a problem because many packages can have 600 to over 2000 calories per container and consumers are eating many of these packages in one sitting.

Particularly preferred fat emulsion compositions are described in more detail below. In one embodiment of the invention a fat composition has improved taste, texture, melt, and mouth feel when compared to shortening, and has a balance of dietary fats plus beneficial essential fatty acids Omega 6 and Omega 3 ALA's, antioxidants, and dietary fiber. Such a fat composition is an aqueous composition containing water, and preferably includes the following components:
a. highly unsaturated liquid edible oil component at 5% (Soybean, Canola Oil, and Sunflower Oil);
b. 2% coconut oil component (medium heat refined coconut oil);
c. fiber component at 35% (Inulin);
d. vanilla powder component on dextrose base at 3%;
e. a thickening component at 5% (Corn Starch);
f. emulsifier component at 1.5% (Lecithin);
g. 48% water component;
h. additional thickening component at 0.3% (xanthan gum); and
i. a natural preservative tocopherols at 0.001% of composition weight

An embodiment of the present invention is a composition comprising: a. highly unsaturated liquid edible oil component at 5% (Soybean Oil, Canola Oil and Sunflower Oil), by weight of the composition; and b. 2% coconut oil component, by weight of the composition. A further embodiment of the present invention additionally comprises c. fiber component at 35% (Inulin), by weight of the composition. Yet a further embodiment further includes d. vanilla powder component on dextrose base at 3%, by weight of said composition.

A further embodiment of the present invention is the above composition that further comprises e. a thickening component at 5% (Corn Starch), by weight of the composition. An additional embodiment further includes f. an emulsifier component at 1.5% (Lecithin), by weight of the composition. Another embodiment further comprises g. 48% water component, by weight of the composition. Yet another embodiment further comprises h. an additional thickening component (xanthan gum) present in an amount of about 0.3%, by weight of the composition. Another embodiment further includes i. a natural preservative (tocopherols) at 0.001% by weight, based on the weight of the composition. Finally, the composition can further include one or more additional components selected from the group consisting of a flavor component, a color component, a preservative component, a stabilizing component, an emulsifier component, a thickening component that includes hydrocolloids including; gelatin, gellan gum, xanthan gum, pectin, carrageenan, cellulose gum, gum arabic, modified starch and combinations thereof, a supplement component that can include organic food acids selected from the group consisting of citric acid, malic acid, lactic acid, fumaric acid, tartaric acid and adipic acid, a nutraceutical component, a vitamin, a mineral, a medicine component or combinations thereof.

The preferred compositions can be made by a method that includes first adding the water component to a mixing and heating vessel, adding slowly the Inulin fiber component, corn starch as a thickening component, vanilla powder on dextrose base as a thickening and flavor component, and xanthan gum as a thickening component while mixing vigorously until homogeneous. To this mixture the highly unsaturated liquid edible oil component (Soybean Oil, Canola Oil, and Sunflower Oil) can be added, followed by addition of the highly saturated oil (coconut oil), and the emulsifier component lecithin. The mixture is continuously mixed until homogeneous, and then the temperature is increased until the composition reaches 150° F. while continuously mixing vigorously for 5 minutes to 30 minutes to completely emulsify the oil in water composition and create the final fat emulsion composition. The composition will slowly thicken during the heating and mixing stage of the process until the ingredients thicken into a homogeneous fat emulsion composition. The temperature can then be lowered while mixing in the tocopherols as a natural preservative and additional flavors, colors, or preservatives can be added while blending with agitation for another 5 minutes.

Another preferred embodiment includes a fat emulsion composition that has improved taste, texture, melt, and mouth feel when compared to partially hydrogenated fat, and has a balance of dietary fats plus beneficial essential fatty acids Omega 6 and Omega 3 ALA's, antioxidants, and dietary fiber. Such a fat composition is a glycerin based composition, and preferably comprises:
a. highly unsaturated liquid edible oil component at 8% (Soybean, Canola Oil, and Sunflower Oil)
b. 3% coconut oil component (medium heat refined coconut oil)
c. fiber component at 50% (Inulin)
d. vanilla powder component on dextrose base at 3%
e. a thickening component at 5% (Corn Starch)
f. emulsifier component at 1.5% (Lecithin)
g. 29.5% glycerin component

An embodiment of the present invention is a composition comprising: a. highly unsaturated liquid edible oil component at 8% (Soybean Oil, Canola Oil and Sunflower Oil), by weight of the composition; and b. 3% of a highly saturated coconut oil component, by weight of the composition. Another embodiment includes the above composition, and further includes c. a fiber component at 50% (Inulin), by weight of the composition. Yet another embodiment further includes d. a vanilla powder component on dextrose base at 3%, by weight of the composition.

An additional embodiment of the present invention is the above composition, which further includes e. a thickening component at 5% (Corn Starch), by weight of the composition. Yet another embodiment further includes f. an emulsifier component at 1.5% (Lecithin), by weight of said composition. Another embodiment further comprises g. 29.5% glycerin component, by weight of said composition. An additional embodiment further comprises the addition of an additional component selected from the group consisting of a flavor component, a color component, a preservative component, a stabilizing component, an emulsifier component, a thickening component that includes hydrocolloids including; gelatin, gellan gum, xanthan gum, pectin, carrageenan, cellulose gum, gum arabic, modified starch and combinations thereof, a supplement component that can include organic food acids selected from the group consisting of citric acid, malic acid, lactic acid, fumaric acid, tartaric acid and adipic acid, a nutraceutical component, a vitamin, a mineral, a medicine component or combinations thereof.

The preferred compositions can be prepared by a method that comprises adding the glycerin component to a mixing and heating vessel, and then adding slowly the Inulin fiber component, corn starch as a thickening component, the vanilla powder on dextrose base as a thickening and flavor component while mixing; and then adding the highly unsaturated liquid edible oil component (Soybean Oil, Canola Oil, and Sunflower Oil), add the highly saturated oil (coconut oil), and the emulsifier component lecithin. The mixture is continuously mixed until it begins to clump, and which point the temperature is increased to until the composition reaches 150° F. while continuously mixing vigorously for 5 minutes to 30 minutes to completely emulsify the oil in glycerin composition and create the final fat emulsion composition. The composition will slowly thicken during the heating and mixing stage of the process until the ingredients thicken into a homogeneous fat emulsion composition. The temperature then can be lowered while mixing in additional flavors, colors, or preservatives while blending with agitation for another 5 minutes.

The composition of the present invention includes an edible oil component present in an amount of from a bout 0.3% to about 80% by weight, preferably from about 1% to about 40%, more preferably from about 3% to about 30%, even more preferably from about 5% to about 20%, and most preferably from about 10% to about 16%, by weight of the fat emulsion composition. This edible oil component makes up part of the group of ingredients that adjust the taste, texture, and improve the melt and mouth feel of the fat composition. While not intending on being bound by any theory of operation, the inventors believe that the interaction of the group of highly unsaturated oils with the coconut oil component creates an improved elasticity within the fat composition that acts similarly to hydrogenated or partially hydrogenated fat. The edible oil component also improves the health characteristic of the fat compositions because it adds monounsaturated and polyunsaturated fats.

When used to formulate a taste masking composition, the emulsion typically will contain far less edible oil, on the order of, for example, from about 0.3% to about 40% an edible oil component, more specifically 2% to 50% of an edible oil component, even more specifically 3% to 25% of an edible oil component, and most specifically 4% to 15% of an edible oil component, by weight of the composition. Any edible oil component can be used in the embodiments. A preferred example of an edible oil component is a blend of canola, soybean oil, and sunflower oil. Non-limiting examples of an edible oil component acceptable for use in the preferred embodiments are those having a low saturated fat content and high unsaturated fat, including monounsaturated and especially polyunsaturated oils. The edible oil component preferably has little or no specific flavor and should be basically bland or somewhat buttery in taste. The edible oils component can be selected from one or more of the following; canola oil, soybean oil, safflower oil, sunflower oil, sesame oil, walnut oil, olive oil, flaxseed oil, chia seed oil, almond oil, corn oil, grape seed oil, peanut oil, other nut oils, and synthesized or reorganized oils, and combinations thereof.

It is preferred that the edible oil component include the above edible oil (or combinations of oils), which are highly unsaturated fat-containing oils, be combined with an edible oil that is highly saturated, such as coconut oil. The coconut oil portion of the edible oil component preferably is present in an amount within the range of from about 0.3% to about 20%, more preferably from about 1% to about 15%, more preferably from about 2% to about 12%, and most preferably from about 3% to about 8%, by weight of the composition. This coconut oil component is an important aspect of the preferred embodiments insofar is it forms a part of the antioxidant flavor profile and unique texture that the fat emulsion composition can provide in foods. While not intending on being bound by any theory of operation, the inventors believe that the blend of the antioxidant flavor components accomplishes the great taste, texture, melt and mouth feel, without the necessity of partially hydrogenated or fully hydrogenated oils. Any medium heat processed coconut oil can be used for the coconut oil component described herein.

The composition of the present invention includes a fiber component within the range of from about 0.001% to about 70%, preferably from about 2% to about 60%, more preferably from about 5% to about 50%, and most preferably from about 10% to about 40%, by weight of the composition. The preferred fiber component for the present invention is Inulin or fructooligiosaccharides. Inulin and fructooligiosaccharides act as a thickening agent and also improve the texture of the fat composition and add beneficial dietary fiber to the composition. In other embodiments, a soluble dietary fiber component consisting of psyllium husk agglomerated with maltodextrin and sprayed with citric acid to improve its solubility can be used as part of, or as the entire dietary fiber component.

Various load rates of dietary fiber can be incorporated in the invention to create improved texture and mouth feel and at certain load rates can provide dietary benefits including promoting a healthy digestion system, controlling blood sugar levels, lowering cholesterol and providing probiotic benefits. The addition of the dietary fiber permits the addition of water or glycerin into the compositions that helps displace sugar and fat within foods that use of our fat composition.

Non-limiting examples of fiber components useful in the embodiments include, but are not limited to naturally derived soluble fiber; naturally derived inulin; inulin extract; synthetic inulin; hydrolysis products of inulin commonly known as fructooligosaccharides, galacto-oligosaccharides, xylooligosaccharides, or oligo derivatives of starch; husks; brans; psyllium; polysaccharides; oligosaccharides; celluloses and derivatives thereof; starches, modified starches, and derivatives of starches; polycarbophil; lignin; arabinogalactans; chitosans; oat fiber; soluble corn fiber; non-digestible corn or wheat dextrin; locust bean gum and derivatives thereof; hydroxypropylmethyl cellulose (HPMC); pectin; and mixtures thereof.

The preferred fiber component for use in the preferred embodiments can be selected from inulin, wheat dextrin, or fructooligiosaccharides. Inulin, wheat dextrin, and fructooligiosaccharides act as a thickening agent and also improve the texture of the fat emulsion structure and add beneficial dietary fiber to the composition. Various load rates of dietary fiber can be incorporated in the composition to create improved texture and at certain load rates can provide dietary benefits including promoting a healthy digestion system, controlling blood sugar levels, and providing probiotic benefits. The addition of the dietary fiber along with the remaining components allow for the addition of water that helps displace sugar within food applications.

Inulin is indigestible by human enzymes ptyalin and amylase, which are designed to digest starch. As a result, inulin passes through much of the digestive system intact. Inulin is a highly effective prebiotic, stimulating the growth of beneficial probiotic bacteria in the gut. Inulin is used in low fat products because of its ability to provide a creamy smooth texture to products. Inulin is a dietary fiber and is believed to activate beneficial good bacteria in the digestive tract. The activation of these bacteria is thought to reduce the risk of bowel cancer. Inulin has a mildly sweet taste, but does not affect blood sugar levels and is recommended for diabetics. Inulin has been clinically proven to increase calcium absorption. The inherent calcium in dairy foods is now an even better source of this bone-building mineral when inulin is added because inulin improves the body's uptake. Plants containing inulin are used to help relieve diabetes mellitus, a condition characterized by hyperglycemia and/or hyperinsulinemia. The embodiments can provide fat emulsions with inulin as the fiber component and calcium supplementation. This fat emulsion would have an improved absorption of calcium because of the inulin within its composition. Calcium is one of the top deficient minerals in the diet of most individuals, and products produced in accordance with the guidelines provided herein can provide great tasting fat emulsions to use in products that can help consumers get more calcium within their diets.

The composition of the present invention also preferably includes a thickening component present in an amount of from about 0.001% to about 35%. A portion of the thickening component can include a thickening component and flavor component that includes a vanilla powder component present in an amount of from about 0% to about 5%, preferably from about 1% to about 4%, and most preferably from about 2% to about 4%, by weight of the composition. This vanilla powder component preferably comes from a vanilla extract sprayed on a dextrose base. The blend of flavor components is another factor in enabling the fat emulsion to achieve great taste, texture, melt and mouth feel, without the necessity for partially hydrogenated or fully hydrogenated fats and high levels of sugar. The vanilla powder component may have a specific flavor profile, and Cook's Flavoring Company currently sells a vanilla powder product that has a suitable flavor profile for use in the present invention. Another vanilla powder or natural vanilla extract could be used, but it should have a similar taste profile and should have the same or similar structure as the Cook's Flavoring Company's vanilla powder.

The thickening component may further comprise other thickeners in addition to the vanilla in amounts within the range of from about 0.001% to about 35%, preferably from about 1% to about 12%, more preferably from about 2% to about 10%, and most preferably from about 3% to about 8%, by weight of the composition. The thickening component can be derived from corn starch, polysaccharides, vegetable gums, and pectin or any combination of these thickening agents. The thickening component helps provide the structure for the fat composition and helps create the rheological characteristics of the final fat composition.

When used to formulate a taste masking composition, the amount of thickening component typically is increased to provide an emulsion that can be hard at room temperature and capable of tableting, milling, forming into a powder, or combined with a liquid and sprayed onto a carrier and optionally milled into a powder. For example, a taste masking composition might contain from about 0.001% to about 70% a thickening component, more specifically 5% to 60% a thickening component, and most specifically 10% to 35% a thickening component, by weight of the composition. In the embodiments, it is preferred to use corn starch as the thickening component.

When used to formulate a taste masking composition, the emulsion also preferably contains flavorants in the form of extracts of various flavors, sugar alcohols to improve the sweetness, and an organic acid component. For example, a taste masking composition might contain from about 0.001% to about 25% of a strawberry extract component, of from about 1% to about 15%, or from about 3% to about 10%, or from about 5% to about 8%, by weight of the composition. The composition also can include from about 0.001% to about 25% a lemon extract component, or from about 1% to about 15%, or from about 3% to about 10%, or from about 4% to about 8%, by weight of the composition. It also is desirable that the composition include from about 0.001% to about 25% liquid vanilla extract, or from about 1% to about 15%, or from about 3% to about 10%, or from about 3.5% to about 8%, by weight of the composition. The sugar alcohol may be present in an amount of from about 0.001% to about 25%, or from about 0.25% to about 15%, or from about 0.5% to about 10%, or from about 1% to about 8%, by weight of the composition, and the organic acid may be present in an amount of from about 0.001% to about 25%, or from about 1% to about 15%, or from about 2% to about 10%, or from about 3% to about 8%, by weight of the composition

The composition also may include an emulsifier component in an amount within the range of from about 0.001% to about 10%, more preferably from about 0.001% to about 5%, more preferably from about 0.001% to about 1%, even more preferably from about 1% to about 3%, and most preferably from about 3% to about 5%, by weight of the composition. When used to formulate a taste masking composition, the amount of emulsifier may be varied to be within the range of from about 0.001% to about 10%, or from about 0.5% to about 8%, of from about 0.75% to about 5%, or from about 1% to about 3%, by weight of the composition. The emulsifier component can come from several different sources and can be any emulsifier commonly utilized in foodstuffs. Preferred embodiments utilize lecithin as the primary emulsifier agent. The emulsifier agent acts as the binding agent keeping the mixture of the oil in water emulsion together and helps make the final fat composition structure. Below is a chart that shows the HLB numbers of some of the most common emulsifiers in the food industry and could be used in certain embodiments of this invention. While this list of emulsifiers contains many of the most common emulsifiers in the food industry other known emulsifiers available and used within the wider food, health and wellness products, skin care industry and other consumer goods categories can be used within the present invention.

Glycol Distearate HLB=1

Sorbitan Trioleate HLB=1.8

Propylene Glycol Isostearate HLB=2.5

Glycol Stearate HLB=2.9

Sorbitan Sesquioleate HLB=3.7

Glyceryl Stearate HLB=3.8

Lecithin HLB=4

Sorbitan Oleate HLB=4.3

Sorbitan Monostearate NF HLB=4.7

Sorbitan Stearate HLB=4.7

Sorbitan Isostearate HLB=4.7

Steareth-2 HLB=4.9

Oleth-2 HLB=4.9

Glyceryl Laurate HLB=5.2

Ceteth-2 HLB=5.3

PEG-30 Dipolyhydroxystearate HLB=5.5

Glyceryl Stearate SE HLB=5.8

Sorbitan Stearate (and) Sucrose Cocoate HLB=6

PEG-4 Dilaurate HLB=6

Methyl Glucose Sesquistearate HLB=6.6

Lecithin HLB (variable)

PEG-8 Dioleate HLB=8

Sorbitan Laurate HLB=8.6

PEG-40 Sorbitan Peroleate HLB=9

Laureth-4 HLB=9.7

PEG-7 Glyceryl Cocoate HLB=10

PEG-20 Almond Glycerides HLB=10

PEG-25 Hydrogenated Castor Oil HLB=10.8

Stearamide MEA HLB=11

Glyceryl Stearate (and) PEG-100 Stearate HLB=11

Polysorbate 85 HLB=11

PEG-7 Olivate HLB=11

Cetearyl Glucoside HLB=11

PEG-8 Oleate HLB=11.6

Polyglyceryl-3 Methyglucose Distearate=12

Oleth-10 HLB=12.4

Oleth-10/Polyoxyl 10 Oleyl Ether NF HLB=12.4

Ceteth-10 HLB=12.9 PEG-8 Laurate HLB=13

Cocamide MEA HLB=13.5

Polysorbate 60 NF HLB=14.9

Polysorbate 60 HLB=14.9

Polysorbate 80 HLB=15

Isosteareth-20 HLB=15

PEG-60 Almond Glycerides HLB=15

Polysorbate 80 NF[HLB=15

PEG-20 Methyl Glucose Sesquistearate HLB=15

Ceteareth-20 HLB=15.2

Oleth-20 HLB=15.3

Steareth-20 HLB=15.3

Steareth-21 HLB=15.5

Ceteth-20 HLB=15.7

Isoceteth-20 HLB=15.7

Polysorbate 20 HLB=16.7

Polysorbate 20 NF HLB=16.7

Laureth-23 HLB=16.9

PEG-100 Stearate HLB=18.8

Steareth-100 HLB=18.8 and

PEG-80 Sorbitan Laurate HLB=19.1

The fat emulsion composition may include a water component present in an amount within the range of from about 1% to about 60%, preferably from about 3% to about 50%, more preferably from about 7% to about 45%, and most preferably from about 15% to about 40%, by weight of the composition. This water component can be derived from any suitable purified potable water source. A different water component may also be derived from an ingredient that has water as its primary component such as fruit juices, fruit flavored drinks, milk, buttermilk, or other source of liquid that contains water as the primary ingredient. The water component adds to the overall texture and melt and chewiness of the flavored chewy or gummy confection. While not intending on being bound by any theory of operation, the inventors have found that by creating an oil in water emulsion and adding the additional components, a fat emulsion composition with a fat continuous phase is created that can have varied viscosities based on the amount of water within the composition.

Embodiments of the invention also include glycerin, especially when used to formulate a taste masking composition. If glycerin is used in addition to water, then it is preferred that glycerin be present in an amount within the range of from about 0.001 to about 15%, more preferably less than 10% and most preferably less than 5% by weight of the composition. Alternatively, if glycerin is used instead of water, as would typically be the case in formulating a taste masking composition, the glycerin component preferably is present in an amount within the range of from about from about 1% to about 40%, of from about 2% to about 40%, or from about 3% to about 20%, or from about 4% to about 10%, by weight of the composition. This glycerin component can be derived from any suitable source well known in the art. The glycerin component adds to the overall texture and melt and chewiness of the fat emulsion composition.

While not intending on being bound by any theory of operation, the inventors have found that by creating oil in glycerin emulsions and adding the additional components, a fat emulsion composition with a fat-like continuous phase is created that can have varied viscosities based on the amount of glycerin and thickeners used within the composition. Depending on the ultimate product to be produced (e.g., a chew, a flowable paste, a powder, a tablet, etc.), an using the guidelines provided herein, a person having ordinary skill in the art will be capable of varying the amount of thickener, fiber, glycerin, etc., to provide the final desired viscosity and compositional structure.

Various embodiments of the fat emulsion compositions or the preferred embodiment also may include at least about 0% or greater, by weight of the composition, of an additional component selected from a flavor component, a color component, a preservative component, a stabilizing component, an emulsifier component, a thickening component, a gelling component like gelatin or pectin, a supplement component that can include a protein source like whey protein isolate or another protein source suitable for processing in a flavored chewy candy or gummy confection, a nutraceutical component, a vitamin, a medicine component or combinations thereof.

Non-limiting examples of such other components include: calcium, potassium, B vitamins, vitamins A, C, D, E, and K, folic acid, other vitamins and minerals commonly known in the art and used for supplementing the diet; extracts and active phytochemicals including ferulic acid (from apples), ginseng, ginko biloba, beta carotene, capsicanoids, anthocyanidins, bioflavinoids, d-limonene, isothiocyanates, cysteines from garlic, ginger, grapes, catechins and polyphenols from teas, onions, phytosterols, isoflavones, lycopene, curcumin, caffeine; glucosamine, chondroitin, msm; melatonin, seratonin; and mixtures thereof.

The compositions described herein also may contain one or more preservatives. Non-limiting examples of suitable preservatives include: sodium benzoate, sodium citrate, sodium phosphate, potassium metabisulfite, sodium metabisulfite, sodium lactate, sodium sulfite, EDTA (ethylenediaminetetraacetic acid), methylparaben, TBHQ, tocopherols, and mixtures thereof.

The compositions also may contain up to 5% by weight of a hydrocolloid component, preferably a strong hydrocolloid component, up to 25% of a protein component, and up to 5% of an acidulant. Certain hydrocolloids like xanthan gum, cellulose, gum aribic, methocel and others known in the food industry can improve the adhesiveness and help reduce oil droplet size within the fat emulsion compositions thereby adding stability to the compositions. Animal based proteins and vegetable based proteins can be used in the fat emulsion to improve stability and improve taste, texture and mouth feel. Illustrative examples of the types of acidulants that may be used in the embodiments include, without limitation, acetic acid, citric acid, hydrochloric acid, lactic acid, malic acid, phosphoric acid, glucono-δ-lactone, mixtures thereof and the like.

When used to formulate a taste masking composition, or when used as a carrier for an active component, the fat emulsion may contain from about 0.001% to about 50% of an active ingredient, or from about 0.5% to about 40%, or from about 0.75% to about 30%, or from about 1% to about 20% an active ingredient selected from pharmaceutical, nutraceutical, over the counter drugs and prescription medicines, by weight of the composition. Any active can be used in the embodiments, and the embodiments have proven useful for use with unpalatable actives, especially those intended to be ingested by humans. Some actives known to have undesirable or unpalatable tastes include, for example, acetaminophen, Acifur, Acyclovir, Alocril, Antabuse, Antithrombin III, ATnativ, Avirax, Azelastine, Azep, castor oil, cod liver oil, cefpodoxime proxetil, Crolon, Cromese and derivatives, Cromolyn and derivatives, Cromoptic, Daraprim, Desloratine, Dextromethorphan, Diethylpropion and derivatives, diphenhydramine hydrochloride, Disulfiriam, docusate sodium, Duromine, Emedastine, Enoxacin, Enoxin, Epivar, Fasigyn, Flagyl, Flexainide, Flecatab, Gastrocram, ibuprofen, Intal, Maloprim, Metrogyl, Metronidazole, Metronide, Mexiletine, Mexitil, Nasalcrom, Nedocromil, Nobesine, Novo-Cromolyn, Opticrom, Oxypentifylline, Phentermine, Rynacrom, Simplotan, sodium cromoglycate, Tambocor, Tenuate, Tenuate Dospan, Tepanil, Tilade, Tinidazole, Trantal, Valaciclovir, Valacyclovir, Valtrex, Zovirax, adipic acid, ascorbic acid, vitamins and minerals, macrolide antibiotics such as clarithromycin, non-steroidal anti-inflammatory drugs, Cefuroxime axetil, Amobarbital, ciprofloxacin hydrochloride, sildenafil citrate, valerian extracts, erythromycin, alprazolam, norfloxacin, ondasetron, granisetron hydrochloride, levofloxacin, clopidogrel sulfate, telithromycin, pristinamycin, guaifenesin, amino acids (L-alanine, L-aspartic acid, etc.), dihydrocodeine phosphate, caffeine, theophylline, fluoxetine, cetirizine hydrochloride, sumatriptan succinate, praziquantel, epsiprantel, ranitidine hydrochloride, risperidone, roxithromycin, diclofenac acid, paracetamol, pioglitazone, donepezil chloride. Preferred actives for use in the embodiments include, acetaminophen, docusate sodium, castor oil, and the like.

Particularly preferred embodiments are described below with respect to fat emulsions that are not specifically adapted for use in masking the taste of undesirable actives and components, but could be used for this purpose. The ingredients used in taste masking compositions, such as glycerin, thickeners, emulsifiers, etc., are the same or similar, except that the product may be made differently, and may include varying amounts of ingredients. An improved health characteristic fat emulsion composition that can provide antioxidant content, reduced sugar content, reduced fat content, beneficial dietary fiber and other dietary benefits to a wide range of foods while maintaining great taste, texture, and mouth feel in the food preferably comprises the following; a. an edible oil component that has a highly unsaturated fat component at 1% to 40%, by weight of the composition; b. an edible oil component that has a highly saturated fat content component at 0.3% to 20%, by weight of the composition; c. a fiber component at 0.001% to 70%, by weight of the composition; d. a vanilla powder component at 0% to 5%, by weight of the composition; e. a thickening agent component at 0.001% to 35%, by weight of the composition; f. an emulsifier component at 0.001% to 5%, by weight of the composition; g. a water component at 1% to 60%, by weight of the composition; a flavor component, a color component, a preservative component, a stabilizing component, an emulsifier component, a thickening component, a gelling component like gelatin or pectin, a supplement component that can include a protein source like whey protein isolate or another protein source suitable for processing in a fat composition, a nutraceutical component, a medicine component or combinations thereof. This fat composition exhibits similar mouth feel within foods when used without the necessity of using negative health characteristics fats such as Trans fats, fully hydrogenated oil, partially hydrogenated oil or high saturated fat content fats like milk fat or animal based fats like lard or tallow.

Another improved health characteristic fat emulsion composition that can provide antioxidant content, reduced sugar content, reduced fat content, beneficial dietary fiber and other dietary benefits to a wide range of foods while maintaining great taste, texture, and mouth feel in the food comprises the following; a. an edible oil component that has a highly unsaturated fat component at 1% to 40%, by weight of the composition; b. an edible oil component that has a highly saturated fat content component at 0.3% to 20%, by weight of the composition; c. a fiber component at 0.001% to 70%, by weight of the composition; d. a vanilla powder component at 0% to 5%, by weight of the component; e. a thickening agent component at 0.001% to 35%, by weight of the composition; f. an emulsifier component at 0.001% to 5%, by weight of the composition; g. a glycerin component at 1% to 60%, by weight of the composition; a flavor component, a color component, a preservative component, a stabilizing component, an emulsifier component, a thickening component, a gelling component like gelatin or pectin, a supplement component that can include a protein source like whey protein isolate or another protein source suitable for processing in a fat composition, a nutraceutical component, a medicine component or combinations thereof. This fat composition exhibits similar mouth feel within foods when used without the necessity of using negative health characteristics fats such as Trans fats, fully hydrogenated oil, partially hydrogenated oil or high saturated fat content fats like milk fat or animal based fats like lard or tallow.

The fat emulsions described above can be used within a wide range of food categories including; 1) milk based foods; ice cream, butter, margarine, cream cheese, cheese, cottage cheese, milk, pudding, coffee creamers, buttermilk, cream, yogurt, kefir; 2) baked goods ready to eat and pre-made mixes that need only to be baked; cookies, cakes, breads, bagels, donuts, crackers, pie crust, pies, chips, pizza dough, nutrition bars, brownies, muffins, cake mixes, cereals, premade baked goods that can be microwave warmed, pastries, snack foods, batters; 3) cake frosting; 4) condiments; salad dressings, mayonnaise, vegetable and chip dips; 5) peanut butter and other nut butters; 6) nutritional drinks; protein based, general nutrition, kids nutrition, weight loss, fiber supplementation, adult nutrition; 7) food bars and snack bars; protein, nutrition, weight loss, energy, granola, fiber supplementation, breakfast, meal replacement, cereal, fruit and grain; 8) breakfast syrups; 9) fruit snacks; 10) syrups and powders used in milk; chocolate, caramel, strawberry, vanilla; 11) Ice cream toppings; caramel, chocolate, butterscotch; 12) pasta noodles; 13) cereals; 14) pre-made food entrees and pre-made snack foods that are sold in the freezer section of grocery; 15) confections; peanut brittle, toffee or butter or glaze covered popcorn and nut clusters, candy coated nuts, caramelized nuts, flavored hard candy, chocolate confections, chocolate coatings, compound coatings, caramel confections, and flavored chewy or gummy confections; and 16) any food that has a fat structure or that has a monosaccharide and disaccharide composition.

It is preferred in the compositions that the edible oil component with a high level of unsaturated fatty acids be present in an amount within the range of from about 1% to about 40%, preferably from about 2% to about 30%, more preferably from about 3% to about 20%, and most preferably from about 5% to about 10%, by weight of the composition. The edible oil component can be selected from the group of edible oils including; canola oil, soybean oil, safflower oil, sunflower oil, sesame oil, walnut oil, olive oil, flaxseed oil, chia seed oil, almond oil, corn oil, grape seed oil, peanut oil, other nut oils, and combinations thereof or other edible oils or synthesized or rearranged oils with similar characteristics that have a high level of unsaturated fatty acids. It is preferred that the edible oil be comprised of a blend of canola, soybean oil, and sunflower oil or canola oil.

It is preferred in the compositions that the edible oil component with a high level of saturated fatty acids be present in an amount within the range of from about 0.3% to about 20%, preferably from about 1% to about 15%, more preferably from about 2% to about 12%, and most preferably from about 3% to about 8%, by weight of the composition. The edible oil component may be selected from the group of edible oils including; coconut oil, palm oil, fractionated coconut or palm oil, partially hydrogenated coconut or palm oil, fully hydrogenated coconut or palm oil, or any other synthesized or altered edible oils including partially hydrogenated oils and fully hydrogenated oils that have either highly saturated or highly unsaturated fatty acids that when hydrogenated become solid similar to coconut oil in consistency including partially hydrogenated soybean oil, cotton seed oil, palm kernel oil or combination of these edible oils. It is especially preferred that the edible oil with highly saturated fatty acid content is coconut oil. Coconut oil has a slight flavor to it and makes up the antioxidant flavor profile that is part of the taste profile. Coconut oil also has a majority of its fatty acids as medium chain fatty acids that are converted into energy and not converted into fat and stored in the body. Coconut oil also has many positive health benefits when compared to other highly saturated fatty acids including that coconut oil is sustainable.

It is preferred in the compositions that the fiber component be present in an amount within the range of from about 0.001% to about 70%, preferably from about 2% to about 60%, more preferably from about 10% to about 50%, and most preferably from about 20% to about 45%, by weight of the composition. The fiber component can be selected from one of the following; naturally derived soluble fiber, naturally derived inulin, inulin extract, synthetic inulin, hydrolysis products of inulin commonly known as fructooligosaccharides, galacto-oligosaccharides, xylooligosaccharides, oligo derivatives of starch; husks; brans; psyllium; polysaccharides, starches; polycarbophil; lignin; arabinogalactans; chitosans; oat fiber; soluble corn fiber, non-digestible corn dextrin; non-digestible wheat dextrin; locust bean gum and derivatives of locust bean gum; hydroxypropylmethyl cellulose (HPMC); pectin; and mixtures thereof. It is preferred that the fiber component be naturally derived inulin or fructooligosaccharides or combinations of them.

It also is preferred in the compositions that vanilla powder component be present in an amount within the range of from about 0% to about 5%, preferably from about 1% to about 4%, and most preferably from about 2% to about 4%, by weight of the composition. The vanilla powder component can be a form of natural vanilla extract sprayed on a dextrose base. This vanilla powder is part of an antioxidant flavor profile that combines with the coconut oil to create a unique flavored fat emulsion taste profile. The dextrose also has thickening properties and helps to create the final viscosity of the fat emulsion composition.

Another preferred embodiment for the compositions described herein is that the thickening component be present in an amount within the range of from about 0.001% to about 35%, preferably from about 1% to about 12%, more preferably from about 2% to about 10%, and most preferably from about 3% to about 8%, by weight of the composition. The thickening component may be derived from polysaccharides, vegetable gums, and pectin or any combination of these thickening agents. It is especially preferred that the thickening component is corn starch.

Another preferred embodiment for the compositions described herein is that the emulsifier component be present in an amount within the range of from about 0.001% to about 5%, preferably from about 0.5% to about 3%, and most preferably from about 1% to about 2%, by weight of the composition. The emulsifier component may be selected from one of the following; lecithin, mono- and diglycerides, modified corn starch, or other suitable emulsifiers known to be used in food production. It is especially preferred that the emulsifier is lecithin.

Another preferred embodiment for the compositions described herein is that the purified water component be present in an amount within the range of from about 1% to about 60%, preferably from about 3% to about 50%, more preferably from about 7% to about 45%, and most preferably from about 15% to about 40%, by weight of the composition. A different water component could come from an ingredient that has water as its primary component such as fruit juices, fruit flavored drinks, milk, buttermilk, or other source of liquid that contains water as the primary component.

The compositions described herein also may be supplemented with an additional component selected from the group consisting of a flavor component preferably natural or could be artificial flavor, a color component, a preservative component, a stabilizing component, an emulsifier component, a thickening component that includes hydrocolloids including; gelatin, gellan gum, xanthan gum, pectin, carrageenan, cellulose gum, gum arabic, modified starch wheat flour or enriched wheat flour or bleached flour or any type of flour from a natural source and combinations thereof, a supplement component that can include organic food acids selected from the group consisting of citric acid, malic acid, lactic acid, fumaric acid, tartaric acid and adipic acid, a nutraceutical component, a medicine component or combinations thereof.

A particularly preferred fat emulsion composition based on water includes the following:
a. highly unsaturated liquid edible oil component at 5% to 10% (Soybean, Canola Oil, and Sunflower Oil or Canola);
b. 2% to 5% coconut oil component;
c. fiber component at 28% to 50% (Inulin);
d. vanilla powder component on dextrose base at 3%;
e. a thickening component at 4% to 8% (Corn Starch);
f. emulsifier component at 2% to 4% (Lecithin);
g. 40% to 55% water component;
h. 0.3% xanthan gum; and
i. a natural preservative tocopherols at 0.001% of composition weight.

Another particularly preferred fat emulsion composition based on glycerin includes the following:
a. highly unsaturated liquid edible oil component at 5% to 10% (Soybean, Canola Oil, and Sunflower Oil or Canola);
b. 2% to 5% coconut oil component;
c. fiber component at 28% to 50% (Inulin);
d. vanilla powder component on dextrose base at 3%;
e. a thickening component at 4% to 8% (Corn Starch);
f. emulsifier component at 2% to 4% (Lecithin);
g. 40% to 55% glycerin component; and
h. a natural preservative tocopherols at 0.001% of composition weight.

Another embodiment of the invention includes a method of preparing a fat emulsion composition that can provide antioxidant content, reduced sugar content, reduce calorie, beneficial dietary fiber and other dietary benefits to a wide range of foods while maintaining great taste, texture, and mouth feel of the product by adding the following ingredients into a cooking vessel: the Inulin fiber component; the corn starch thickening component; the vanilla powder on a dextrose base component; and mixing them into the purified water slowly until homogeneous. The method continues by adding the highly unsaturated fat component (Soybean Oil, Canola Oil, Sunflower Oil) the highly saturated fat component (coconut oil), the emulsifier (lecithin) component, and the xanthan gum component and blending until homogeneous. The mixture will now look like a viscous pourable fat composition that moves similar to a fat made with soft room temperature edible oils. The homogeneous mixture then is brought to a temperature of about 150° F. and continuously mixed until the thickening emulsion process occurs. This thickening of the emulsion usually occurs within a time period of from about 5 minutes to about 30 minutes of mixing, although it could take longer depending on the heating and mixing vessel, the atmospheric pressure, and the concentration of ingredients. A person having ordinary skill in the art will be capable of determining when the thickening emulsion process is completed and can adjust the temperature and time of heating accordingly.

Once the emulsification occurs, the mixture is continuously stirred, and the mixing and heating vessel is removed from the heat source. Upon removal from the heat source, the tocopherols natural preservative component is added. The fat emulsion will be sufficiently hardened at this point to be capable of being scooped with a spoon or other scooping device and placed into a mold or other sufficient storage device to allow the fat composition to continue to cool. Once completely cooled, the fat composition will have the viscosity similar to Crisco™brand shortening. A softer, thinner, and less viscous fat can be made by having a higher level of water in the composition or by having a lower level of the Inulin fiber component. A harder, thicker, and more viscous fat can be made by lowering the level of water in the composition or by adding a higher percentage of the Inulin fiber component. Those having ordinary skill in the art will be capable of adding sufficient quantities of components to provide the desired final product consistency and viscosity, using the guidelines provided herein.

Within certain embodiments of this invention, the composition may have a viscosity such that the fat emulsion composition can replace partially hydrogenated oil, fully hydrogenated oil, and Trans Fat. This can be accomplished by adjusting the amounts of the Inulin fiber component and the water component within the fat composition. A healthy fat emulsion composition that can replace partially hydrogenated oil, fully hydrogenated oil, and Trans Fat can have a profound effect on improving consumer health because the fat emulsion compositions described herein have zero Trans Fats, and the hardness of the fat composition comes from a healthy interaction of dietary fiber, water, thickening components, and edible oils and not from hardened fatty acids that are proven to cause negative health problems like cardiovascular disease and heart disease. The most efficient and least costly way to produce the above-described fat emulsion compositions would be to produce them in a large commercial processing line that can mix, heat to emulsify, and package the fat composition in a continuous processing system.

Another preferred embodiment of the invention includes a method of preparing a fat emulsion composition that can provide that can provide antioxidant content, reduced sugar content, beneficial dietary fiber and other dietary benefits to a wide range of foods while maintaining great taste, texture, and mouth feel of the product by adding the following ingredients into a cooking vessel: the Inulin fiber component; the corn starch thickening component; and the vanilla powder on a dextrose base component and mixing them into the glycerin slowly. While mixing, the highly unsaturated fat component (Soybean Oil, Canola Oil, Sunflower Oil) the highly saturated fat component (coconut oil), the emulsifier (lecithin) component are added and blended into the mixture. The mixture will now look like a loose group of ingredients that are starting to bind together. The mixture then is heated to a temperature of 150° F. and continuously mixed until the thickening emulsion process occurs. This thickening of the emulsion usually occurs in about 5 minutes to about 30 minutes of mixing, although it could take longer depending on the heating and mixing vessel, the atmospheric pressure, and the concentration of ingredients. A person having ordinary skill in the art will be capable of determining when the thickening emulsion process is completed and can adjust the temperature and time of heating accordingly.

Once the emulsification occurs, the mixture is continuously stirred, and the mixing and heating vessel is removed from the heat source. Upon removal from the heat source, natural preservatives or other additional components may optionally be added. The fat emulsion will be sufficiently hardened at this point to be capable of being scooped with a spoon or other scooping device and placed into a mold or other sufficient storage device to allow the fat composition to continue to cool. Once completely cooled, the fat composition will have the viscosity similar to partially hydrogenated oil. A softer, thinner, and less viscous fat emulsion can be made by utilizing a higher level of glycerin in the composition or by using less inulin fiber component. A harder, thicker, and more viscous fat emulsion can be made by lowering the level of glycerin in the composition or by adding a higher percentage of the inulin fiber component.

Within certain embodiments of this invention, the composition may have a viscosity such that the fat emulsion composition can replace partially hydrogenated oil, fully hydrogenated oil, and Trans Fat. This can be accomplished by adjusting the amounts of the Inulin fiber component and the glycerin component within the fat composition. A healthy fat emulsion composition that can replace partially hydrogenated oil, fully hydrogenated oil, and Trans Fat can have a profound effect on improving consumer health because the fat emulsion compositions described herein have zero Trans Fats, and the hardness of the fat composition is derived from a healthy interaction of dietary fiber, glycerin, thickening components, and edible oils, and not from hardened fatty acids that are proven to cause negative health problems like cardiovascular disease and heart disease. The most efficient and least costly way to produce the above fat emulsion composition would be to produce the fat emulsion composition in a large commercial processing line that can mix, heat to emulsify, and package the fat composition in a continuous processing system.

The methods described above for the water-based and glycerin-based fat emulsion composition also may include the addition of one or more additional components to the fat emulsion composition. Suitable additional components may be selected from the group consisting of; a flavor component, a color component, a preservative component, a stabilizing component, an emulsifier component, a thickening component, a supplement component, a protein component, a nutraceutical component, a vitamin component, a medicine component or combinations thereof. These additional ingredients would be added at the appropriate point above within the processing of the fat composition which means some would be added within the mixing stage (before heating to about 150° F.) and some would be added once the temperature of the composition is lowered to around 100° F. (after heating to about 150° F.), and blended into the fat composition.

Another embodiment of the fat emulsion described herein include a fat emulsion composition that includes an edible oils component of from about 0.3% to about 80%, a dietary fiber component from about 0.001% to about 70%, a thickening component from about 0.001% to about 35%, a water component or glycerin component from about 1% to about 60%, an emulsifier component from about 0.001% to about 10%, a glycerin component from about 0% to about 15% (if water is used), a strong hydrocolloid structure from about 0% to about 5%, a protein component from about 0% to about 25%, and an acidulant component from about 0% to about 5% all as a percentage of the weight of the composition. The composition also may contain an additional component selected from a flavor component, a color component, a preservative component, a stabilizing component, a gelling component like gelatin or pectin, a supplement component, a nutraceutical component, a medicine component or combinations thereof.

It is preferred in the embodiments that the composition contain from about 0.3% to about 80%, more preferably from about 1% to about 70%, more preferably from about 2% to about 60%, even more preferably from about 2.2% to about 50%, and most preferably from about 2.5% to about 40% of an edible oils component, based on the total weight of the composition. The edible fat or oil may include those fats or oils that are liquid at room temperature and that are hard at room temperature. The edible oil component can be selected from traditional fats or oils, from inter-esterfied fats or oils, from hydrogenated fats or oils, or from fats or oils that have been physically rearranged to create designer fat or oil structures. The edible fat or oil component also may be a fat substitute in lieu of oil or in combination with oil; conventional fat substitutes may be used. Preferred fat substitutes employable in this invention include fatty acid-esterified alkoxylated glycerin compositions as well as sucrose fatty acid esters. The preferred edible oil used in the present invention is an oil/fat that is liquid at room temperature like; canola oil, sunflower oil, soybean oil, olive oil, almond oil, safflower oil, and other oils that have a predominant unsaturated fat content within them or the edible oil can be a combination of a oil/fat that is liquid at room temperature like canola oil as the predominant oil/fat and a lower component of a solid at room temperature oil like; coconut oil, cocoa butter, shea butter or another suitable solid at room temperature oil/fat. In cases where the oil/fat content is both a liquid oil and solid oil/fat at room temperature the ratio would be from 10:1 to 2:1 liquid oil/fat vs. solid oil/fat.

The fat emulsions described above preferably have very low fat content from about 0.3% to about 10% fat content and the viscosities of the final fat emulsion products can be designed to have a range from a pourable structure at room temperature, similar to cream or the fat structure necessary to make pourable salad dressings, spoonable viscosities similar to the fat structure necessary to make mayonnaise or dips, spreadable viscosities similar to the fat structure necessary to make margarine, butter, or cheese spreads, scoopable viscosities similar to the fat structure necessary to make shortening or similar to the group of fats that make up palm oil, and more viscous scoupable viscosities similar to the fat structure that makes up fats once they are hydrogenated and the structure of the synthesized ingredient Trans Fat.

The low fat content 0.3% to 10% emulsions that can be created above also may have similar rheological characteristics to the previously described pourable at room temperature structure fats that can be used to make cream or salad dressings which are a rich and creamy mouth feel, smooth texture, and soft thickened fat taste. The low fat content emulsions that can be created within this invention have a very smooth fat taste that lasts for only a couple of seconds in the palate and then becomes a hint of sweetness leftover in the mouth.

The more viscous spoonable low fat content 0.3% to 10% emulsions have a smooth consistency and mouth feel that has a longer lasting melt within the mouth and takes two to four seconds to clear the palate also with a slight sweetness leftover. The scoopable more viscous low fat content 0.3 to 10% emulsions have a rich and creamy mouth feel that has the same characteristics of shortening or palm oil in that the consistency of the emulsion in the mouth has a pronounced fat texture and taste. The scoopable more viscous low fat content 0.3% to 10% emulsion produces the same “feel properties as shortening and palm oil”, i.e.: it produces a greasy feeling when rubbed between the fingers as does shortening and palm oil. The residue on the fingers coats the surface of the skin in the same way as shortening and palm oil and it is difficult to wash off as is shortening or palm oil. The scoopable low fat content 0.3 to 10% emulsions that can be made with this invention melt in the mouth over a longer time period versus the spoonable low fat content 0.3% to 10% emulsions. It takes five to over seven seconds for the scoopable viscosity low fat content 0.3 to 10% emulsion to completely clear the palate with a slight sweetness leftover. All viscosities of the low fat content 0.3% to 10% emulsions have a pleasant taste profile that is slightly sweet and that leaves a pleasant aftertaste in the mouth once completely out of the palate.

The medium to more viscous low fat 0.3% to 10% emulsions that can be created in accordance with the preferred embodiments have a very plastic structure to them. They hold their shape extremely well at room temperature and at the most viscous structures actually have bounce back that occurs when the shape of the fat emulsion is changed by low level force. The plastic nature of the low fat 0.3% to 10% emulsions in some of the embodiments patterns the same increase in plasticity that occurs in fats that are used in spoonable food products like margarine and butter, to harder fats like fully hydrogenated fat or Trans Fat. As the inventive low fat 0.3% to 10% emulsions become more viscous the plastic characteristics increase. Hydrogenated fats and Trans Fat have a chewy elastic characteristic and intense mouth feel to them and the inventive low fat 0.3% to 10% emulsion's have similar chewy elastic and intense mouth feel characteristics.

The composition described herein can be added to foodstuffs to create food products that have a fat content from 10% to 80%. Fat emulsions created with the present invention can use liquid at room temperature fat/oil as the primary component. This allows a liquid at room temperate edible oil that is low in saturated fat to exhibit a hardened fat structure without the addition of hydrogenated fats like shortening, or without going through the hydrogenation process. As the fat content increases to the range of 30% or higher, the ability to utilize the inventive fat emulsions described herein to deliver beneficial unsaturated fats to foods, as opposed to the saturated fats which are replaced, is an extremely positive health benefit.

Currently in order to create a hardened fat structure, the fat/oil must be high in saturated fat like palm oil, milk fat, lard or coconut oil, or must be inter-esterfied which means the fats are rearranged to attach saturated fat to softer unsaturated fats to create a hardened fat. In addition, creating a hardened fat typically requires the use of a hydrogenated fat as a component of the fat like shortening, or the hydrogenated fat must go through the hydrogenation process that renders a liquid or harder oil into a solid, or the fat must be Trans Fat which is a synthetically hardened fat. The embodiments described herein allow liquid vegetable oils that have very low saturated fat levels to become hardened by being incorporated into the fat emulsion. These liquid at room temperature oils, like; canola, soybean, almond oil, and others become hardened at room temperature, when they are incorporated into the inventive fat emulsions described herein. This hardening of the liquid at room temperature edible oil that can be the primary fat/oil component within the structure of the fat emulsion is a unique characteristic of this invention. The ability to turn a liquid edible oil at room temperature into a hardened fat emulsion with a viscosity that is up to 10 times greater, up to 20 times greater, and up to 30 times greater and greater than 30 times greater, when compared to the liquid edible oil at room temperature is an important feature of the embodiments.

The fat emulsion compositions may have a dietary fiber component present in an amount of from about 0.001% to about 70%, more preferably from about 5% to about 60%, more preferably from about 15% to about 50%, even more preferably from about 25% to about 45%, and most preferably from about 30% to about 40%, based on the weight of the composition. The dietary fiber can be any suitable dietary fiber suitable for use in food products. This includes all forms of dietary fiber both soluble and insoluble. Inulin is the preferred soluble dietary fiber source for this fat emulsion invention and creates a consistency from pasty to a smooth and fat-like consistency depending on the micron size of the Inulin used. Inulin that is processed into micron sizes smaller than 149 microns when incorporated the fat emulsion composition, results in a pastier and less smooth texture mouth feel fat emulsion. Inulin processed into micron sizes larger than 149 microns typically will create a smoother texture mouth feel fat emulsion that can mimic the mouth feel of traditional fats. The Micron size of P&G's Clear & Natural Inulin supplement is in the range of 149 microns and larger and is a good Inulin mill size for the present invention, to create fat emulsions that mimic taste, texture and mouth feel of a full fat. Using the guidelines provided herein, those skilled in the art will be capable of utilizing a sufficiently sized inulin fiber to provide the requisite product attributes.

Some suitable forms of dietary fiber that are made up of completely insoluble dietary fiber include many fruit and vegetable skins including tomatoes, apples, plums, legumes such as soybean skins, grains such as flax seed and chia seed, and wood pulps. When insoluble dietary fiber is part of the dietary fiber component or the whole component within the present invention the final fat emulsion would be a coarse emulsion. A coarse fat emulsion created by using insoluble dietary fiber within the present invention could be used in certain food applications that do not require a smooth fat-like composition. A coarse fat emulsion created from the use of insoluble dietary fiber could be further processed utilizing high pressure homogenization or other heat and high shear processing methods to refine the coarse fat emulsion into a smoother more uniform texture fat emulsion. This is desirable because Insoluble dietary fiber provides a range of health benefits to humans, including speeding the transit of food through the digestive tract, promoting regularity, and alleviating constipation.

Corn starch is the preferred thickening component useful in these embodiments, and can be used in an amount within the range of from about 0.001% to about 20%, more preferably from about 1% to about 15%, and most preferably from about 5% to about 10%, based on the total weight of the fat emulsion composition. Corn starch is the preferred thickening component and Inulin is the preferred dietary fiber component for the fat emulsion composition because they both exhibit improved swelling characteristics when heated at temperatures of 150 to 160° F. for 5 minutes to 30 minutes or greater. Shear also has the effect of increasing the swelling capacity of corn starch and Inulin and mixing with a whisk under intense shear or using a heated mixing vessel with a whisk attachment or a homogenization process that applies temperature at 150 to 160° F. under pressure and/or shear is the preferred manufacturing method. The least involved method of processing this fat emulsion composition is to first add the water or glycerin component, then add the dietary fiber and corn starch component followed by the edible oil/fat component and emulsifier component. The mixture would then have its temperature increased to about 150° F. and mixed under shear for about 5 to about 30 plus minutes until it becomes a homogeneous fat emulsion structure. Those skilled in the art will appreciate that the time for mixing during heating may be extended, depending on whether the mixture has emulsified. While not intending on being bound by any theory of operation, the inventors believe that raising temperature to within the range of from about 150 degrees to about 160° F. and introducing shear causes the Inulin and Corn Starch components to thicken and then the structure starts to change and become more viscous as the swelling of the Inulin and Corn Starch continues to stabilize the water (and/or glycerin) content within the composition. At a certain point the composition visually changes and takes on the appearance of a fat more than just a group of mixed ingredients. Once the visual change occurs, the mixing of the fat emulsion must be intensified as the structure continues to take on the characteristics of a pourable fat similar to cream all the way to a scoopable fat similar to fully hydrogenated fat. The continued mixing under shear at the point of the visual structure change to a fat appearance allows the particle size of the emulsion to decrease and improves the final stability of the finished fat emulsion structures. Mixing under intense shear is known to reduce the particle size of fat emulsions and create improved stabilities. The ability of the present invention to utilize a group of ingredients that normally would not produce a viscous fat emulsion under normal mixing and temperature processing methods to create a wide range of stable fat emulsions that have wide range of viscosities and physical characteristics that mimic different fat structures is a desirable property of this invention.

In a preferred embodiment, the composition may include an emulsifier or surfactant in an amount of from about 0.001% to about 10%, more preferably from about 0.5% to about 5%, and most preferably from about 0.8% to about 3%, based on the weight of the composition. The present invention can create fat emulsions with viscosities that range from pourable at room temperature to solid viscous plastic characteristics at room temperature. The fat emulsions created within the present invention require an emulsifier similar in HLB value to lecithin for many of the viscosities. Generally, low HLB emulsifiers are used when the fat emulsion structure has a predominant oil phase and a less dominant water phase and this reverses to the necessity for a higher HLB number emulsifier as the fat emulsion has a predominant water phase and a less dominant oil phase. The HLB number of some of the most common food category emulsifiers is referenced above within the preferred embodiment section. Those skilled in the art of creating fat emulsions have a much wider range of emulsifiers available and any emulsifier suitable for making fat emulsions would be covered within the present invention.

One of the preferred properties of the fat emulsions described above is that the embodiments can provide pourable fat emulsion structures and medium viscosity spoonable fat structures as well as more viscous scoopable fat structures that use HLB number emulsifiers much lower in the scale, when compared to. traditional fat emulsions that have a predominate water phase, and when compared to oil/fat phase. The embodiments enable the use of lecithin as the emulsifier because the water phase becomes stabilized by the dietary fiber component and starch component swelling and becoming a part of the fat emulsion structure. The ability to create such pourable fat emulsion structure, spoonable fat emulsion structure and scoopable fat emulsion structure using lecithin rather than the higher HLB emulsifiers at the 10 plus HLB value is a significant advantage because lecithin is a commonly recognized emulsifier and is viewed as a more consumer friendly ingredient when compared to the 10 plus HLB emulsifier numbers. Consumers are often leery of complicated and unfamiliar ingredient labels and are concerned by complicated chemical names. Lecithin is one of the most widely used emulsifiers and consumers are familiar and comfortable with lecithin as part of an ingredient label. Another method of increasing stability for fat emulsions that can be created in the embodiments herein is the addition of an acidulant to inhibit microbiological growth. When acidified, the edible fat emulsion typically would have enough acidulant added so that the pH of the composition is from about 2.75 to about 5.75, and preferably, from about 2.85 to about 5.50, and most preferably, from about 3.25 to about 4.25, including all ranges subsumed therein. Illustrative examples of the types of acidulants which may be used in this invention include, without limitation, acetic acid, citric acid, hydrochloric acid, lactic acid, malic acid, phosphoric acid, glucono-delta-lactone, mixtures thereof and the like.

Water activity also can be controlled within the inventive fat emulsions by using glycerin as an ingredient within certain embodiments of this invention. Glycerin has a larger molecule size than water and can be added to fat emulsions created within this invention to control water activity and trap water molecules within the fat emulsion system. The present embodiments can utilize glycerin as a nutraceutical component in the range of from about 0% to about 15% by weight of the composition to help control water activity. The ability to utilize glycerin with the present emulsion can be especially important when the fat emulsion is being used in a food system that has a cereal, crisp cookie, cake or other low water activity ingredient in direct contact with the fat emulsion system or where the fat emulsion system is used within the center of a cookie or filling in a snack cake, etc. The ability to prevent migration of water from the emulsion to other ingredients within food and confection applications is an important property of the present invention, and allows for use of the emulsion in a very wide range of food and confection applications.

Protein also can provide stability in oil/fat in water emulsions or water in oil/fat emulsions that can be created in the preferred embodiments. Protein has the characteristic of improving stability in fat emulsions by improvement in adhesiveness and reducing oil droplet size within an emulsion. The improved adhesiveness and reduction of oil droplet size results in improved fat emulsion stability. Animal based proteins and vegetable based proteins can be used within the current fat emulsion invention to improve stability and improve taste, texture and mouth feel. A property of the fat emulsions described herein is that lower levels of protein would be required to create the same improved stability characteristics because the water phase has been stabilized by the dietary fiber and starch, thickening agent components. This is an advantage when compared to other fat emulsion structures because protein can be an expensive component when used to stabilize the structure.

Certain hydrocolloids such as xanthan gum, cellulose, gum aribic, methocel and others known in the food industry can improve the adhesiveness and help reduce oil droplet size within fat emulsion systems and could be used within fat emulsions to add additional stability. Cellulose as a preferred hydrocolloid can provide oil in water fat emulsions that utilize a lower level of the inulin dietary fiber component in order to create the same low fat 0.3% to 10% fat content component fat emulsion to a 10% to 80% fat content fat emulsion. The cellulose ingredient can be supplied by Dow Wolff Cellulosics under the brand name ForteFiber™. Other forms of gums and hydrocolloid ingredients also are available from this company and potentially could be used.

Certain foods such as ice cream, milk, yogurt, baked goods, meats, poultry, and eggs and other food categories that are eaten in larger quantities may require a lower dietary fiber content as part of the fat emulsion structure, so that dietary fiber intake guidelines are not exceeded and the consumer can enjoy these products in the normal quantities they eat. The preferred embodiments allow for the use of cellulose as a hydrocolloid to address this issue and allows for the inulin dietary fiber contents to be appropriate for foods that consumers eat in larger quantities. The flexibility of the inventive fat emulsion to deliver a wide range of dietary fiber content options within its structure is a beneficial property of the invention. Dietary fiber can be included in the preferred compositions in an amount within the range of from about 0.001% to about 70%. This allows for the finished fat emulsion product to have a dietary fiber content that contributes from a gram of dietary fiber to upwards of 50% dietary fiber per customary serving of the food.

The present invention can create fat emulsions that are suitable for shallow frying, pan frying, sautéing, stir frying or any other form of pan cooking that uses a fat/oil source or fat emulsion. The fat emulsions are pourable or squeezable products as evidenced by a Bostwick value of equal to or more than about 7, preferably from about 7 to about 10, and even as high as from about 15 to about 23. Food products that can be created within this invention can have a much higher water phase vs. the oil/fat phase because of the stabilization of the water activity that occurs because of the incorporation of dietary fiber and corn starch and optionally hydrocolloid binders like cellulose, microcrystalline cellulose and carboxymethyl cellulose that can be used to further stabilize water activity, when compared to typical fat emulsions used in frying applications. A low fat frying emulsion product that has water as a high percentage of the overall composition is thus a possible application with extremely low fat contents versus regular frying fat emulsions or fats within certain embodiments of this invention as such embodiments will not exhibit the normal spattering problem found with typical high water content fat emulsions when used for frying.

The fat emulsions of the preferred embodiments may have specific ratios of ingredients that include; water, dietary fiber, essential fatty acids, corn starch or other starch component, protein matrix, and specific nutrients, vitamins and minerals necessary to provide a complete nutritional food system. This complete nutritional food system can be delivered in a very palatable tasting fat emulsion that can be flavored and added to many forms of foods known to be delivery methods for complete nutrition systems including drinks, shakes, nutrition beverages, nutrition bars, supplement delivery forms like chews or other confections or the fat emulsion complete nutritional system can be used for tube feeding. The present invention provides the desirable ability to create fat emulsions that control all the dietary components necessary to make a complete nutritional food.

Instead of being used as a supplement to water, glycerin can be used as a complete replacement to water. When used to replace water, the amount of glycerin typically is present in an amount within the range of from about 1% to about 70%, preferably from about 5% to about 50%, more preferably from about 10% to about 40%, and most preferably from about 15% to about 35%, by weight of the composition. The invention can create oil in glycerin fat emulsions that can have a wide range of viscosities similar to the aqueous-based fat emulsions. The emulsifier necessary to create oil in glycerin fat emulsions does not necessarily follow the same principles as oil in water or fat emulsions made with an aqueous phase. Normally, as the oil phase increases versus the water phase the emulsifier would be a lower number on the HLB scale. Research has shown that oil-in-glycerin, stearic acid-based emulsifiers create the strongest and most stable structure fat emulsions. See Petersen, R. V., et al., “Studies on Nonaqueous Emulsions,” J. Soc. Cosmetic Chemists, Vol. 19, pp. 627-640 (1968).

Particularly preferred fat emulsion taste masking compositions are described in more detail below. The taste masking compositions can be used to mask the taste of unpalatable or undesirable tasting actives that are intended for ingestion by, for example, humans and animals. Young children and elderly patients sometimes have particular difficulty in ingesting poor, bitter, or otherwise unpalatable actives and wellness ingredients. In addition, many animals, such as pets and zoo animals, refuse to ingest unpleasant tasting actives. The fat emulsions described herein are capable of masking the taste of these ingredients, and are capable of providing a suitable delivery vehicle for the actives.

In one embodiment, the fat emulsion masks the taste of docusate sodium, a very unpleasant tasting over the counter medicine stool softener/laxative category active. The ability to mask docusate sodium and create a flowable powder that can be mixed into a solvent like; water, juice, milk, etc. is a unique breakthrough for the industry in the docusate sodium delivery methods. Many of the laxatives and stool softener actives are sold in gel caps, tablets, or flavored liquids and are not very convenient for consumers to ingest. Docusate sodium to date has never been able to be masked well enough to create a more convenient delivery method. The fat emulsion composition described herein, however, is capable of effectively masking the taste of docusate sodium. In a preferred embodiment, the fat emulsion can be milled into a powder, or can be present in an aqueous composition containing water and glycerin, and is described in Example 9 below.

Therefore, a specific embodiment is its ability to deliver therapeutic levels of unpleasant tasting actives, such as docusate sodium, in a very good tasting mixable powder form. Other embodiments of the present invention can create fat emulsions that can deliver therapeutic levels of sodium docusate in a gummy, chew, or other unit dosing formats. The above docusate sodium fat emulsion provides an example of a preferred embodiment of the present invention, but does not by any means limit the scope of the invention. Other active ingredients from the category of pharmaceutical, nutraceutical, over the counter drugs and prescription medicines can be delivered by the present invention. The preferred embodiments described herein can create a wide range of different viscosity fat emulsion compositions based on varying of certain ingredients to provide delivery methods for many difficult to mask active ingredients. The unique structure of the fat emulsion allows for positive health benefits to be delivered to the consumer while being essentially sugar free and low in fat.

### EXAMPLES

The below examples are included for illustrative purposes only and are not intended to in any way limit the scope of the present invention.

### Example 1

This example provides a fat emulsion composition that can be used in foods that have a fluid more water continuous phase like salad dressings, chocolate or caramel syrups, breakfast syrups, or other foods that need a more fluid type of liquid edible oil component.

The following ingredients were added into a mixing and heating vessel; 52# of purified water, then slowly add while mixing 30# of Inulin (dietary fiber component) 7# of corn starch a thickening component, 2# vanilla powder on dextrose base (a flavor and thickening component mix these ingredients until they become homogeneous. Then, about 5# of a liquid edible vegetable oil blend (Soybean Oil, Canola Oil, and Sunflower Oil), sold by Crisco, 2# medium refined coconut oil, 1.5# lecithin (an emulsifier), and 0.3# xanthan gum (an additional thickening agent) were added and all ingredients were mixed together until they became a viscous composition. The complete group of ingredients then were heated to 150° F. while being mixed vigorously for 5 to 30 minutes in order to completely emulsify the ingredients.

During the heating stage the mixture began to thicken into a more viscous more solid looking fat emulsion composition. Once this thickened more viscous stage was achieved, the fat emulsion composition then was slowly cooled and the tocopherols a natural preservative or a stronger preservative was added to the fat emulsion composition and mixed for 10 minutes to completely blend in the preservative component. The cooling fat emulsion composition was then placed into molds or packaged into containers or directly used in the food that will utilize the fat composition. This type of process could be extrapolated into large scale production.

### Example 2

This example provides a fat emulsion composition that can be used in foods that have a fluid more water continuous phase like salad dressings, chocolate or caramel syrups, breakfast syrups, or other foods that need a more fluid type of liquid edible oil component.

The following ingredients were added into a mixing and heating vessel: 52# of glycerin, then slowly add while mixing 30# of Inulin (dietary fiber component) 7# of corn starch a thickening component, 2# vanilla powder on dextrose base (a flavor and thickening component). The ingredients were mixed until they became homogeneous. Then 5# of a liquid edible vegetable oil blend (Soybean Oil, Canola Oil, and Sunflower Oil), sold by Crisco, 2# medium refined coconut oil, and 1.5# lecithin (an emulsifier) were added while continuously mixing. The mixture was then brought up to a temperature of 150° F. while continuously mixed vigorously for about 5 to 30 minutes in order to completely emulsify the ingredients. During the heating stage the mixture started to thicken into a more viscous fat emulsion composition. Once this thickened more viscous stage had been achieved, the fat emulsion composition was taken off the heat and slowly cooled. The cooling fat emulsion composition then was placed into a storage container or packaged into containers or directly used in the food that will utilize the fat composition. This type of process could be extrapolated into large scale production.

### Example 3

This example provides a fat composition that can replace shortening within baked goods, doughs, snack foods, and other foods that use shortening as an ingredient. The following ingredients were added into a mixing and heating vessel; 26# of purified water, 8# of glycerin, then slowly add while mixing 32# of Inulin (dietary fiber component) 18# of corn starch a thickening component, 3# vanilla powder on dextrose base (a flavor and thickening component). The ingredients were mixed until they became homogeneous. Then, 8# of a liquid edible vegetable oil blend (Soybean Oil, Canola Oil, and Sunflower Oil), sold by Crisco, 3# medium refined coconut oil, 1.5# lecithin (an emulsifier), and 0.3# xanthan gum (an additional thickening agent) were added and the ingredients were mixed together until they became a viscous homogeneous composition. The complete group of ingredients were then brought up to 150° F. while being mixed vigorously for about 5 to about 30 minutes in order to completely emulsify the ingredients. During the heating stage the mixture began to thicken into a more viscous, more solid looking fat emulsion composition. Once this thickened more viscous stage had been achieved, the fat emulsion composition was then removed from the heat, slowly cooled, and the tocopherols (a natural preservative) or a stronger preservative was added to the fat composition and mixed for 10 minutes to completely blend in the preservative component. The cooled fat emulsion composition was then placed into molds or packaged into containers or directly used in the food that will utilize the fat emulsion composition. This type of process could be extrapolated into large scale production.

### Example 4

This example provides a fat emulsion composition that can be used as a replacement for partially or fully hydrogenated oils in food products that currently use the oils for taste, texture, mouth feel like; cookies, cakes, coffee creamers, pudding, cake icing, nutrition and snack bars, confections and other foods that use these partially and fully hydrogenated oils except for chocolate.

The following ingredients were added into a mixing and heating vessel; 26# of glycerin, then slowly add while mixing 35.5# of Inulin (dietary fiber component) 23# of corn starch a thickening component, 3# vanilla powder on dextrose base (a flavor and thickening component), and the ingredients were mixed until they became homogeneous. Then, 8# of a liquid edible vegetable oil blend (Soybean Oil, Canola Oil, and Sunflower Oil), sold by Crisco, 3# medium refined coconut oil, and 1.5# lecithin (an emulsifier) were added, and the ingredients mixed together until they became a viscous clump composition. The complete group of ingredients was then brought up to 150° F. while being mixed vigorously for about 5 to about 30 minutes in order to completely blend and emulsify the ingredients. During the heating stage, the mixture began to thicken into a more viscous more solid looking fat emulsion composition. Once this thickened more viscous stage had been achieved, the fat emulsion composition was removed from the heat and slowly cooled. The cooled fat emulsion composition was then placed into molds or packaged into containers or directly used in the food that will utilize the fat emulsion composition. This type of process could be extrapolated into large scale production.

### Example 5

This example provides a fat composition that can be used as a replacement for Trans Fat in food products that currently use it for taste, texture, mouth feel like; cookies and certain other food products. This fat composition can also be added back into foods that have taken Trans Fat out of their compositions because of the negative health affects that Trans Fat is proven to cause including cardiovascular and heart disease.

The following ingredients were added into a mixing and heating vessel; 24# of glycerin, then slowly add while mixing 50# of Inulin (dietary fiber component) 8.5# of corn starch a thickening component, 3.7# vanilla powder on dextrose base (a flavor and thickening component), and mixed until they became homogeneous. Then, 10# of a liquid edible vegetable oil blend (Soybean Oil, Canola Oil, and Sunflower Oil), sold by Crisco, 2# medium refined coconut oil, and 1.8# lecithin (an emulsifier) were added and the ingredients were mixed together until they became a viscous homogeneous composition. The complete group of ingredients was then brought up to 150° F. to 160° F. while being mixed vigorously for about 5 to about 30 minutes in order to completely emulsify the ingredients. During the heating stage, the mixture began to thicken into a more viscous more solid looking fat emulsion composition. Once this thickened more viscous stage had been achieved, the fat emulsion composition was removed from heat and slowly cooled. The cooled fat emulsion composition was then placed into molds or packaged into containers or directly used in the food that will utilize the fat composition. This type of process could be extrapolated into large scale production.

### Example 6

This example provides for a Milk Chocolate Confectionery Coating with Improved Nutritional Structure that includes lower saturated fat, lower sugar, lower total fat and beneficial dietary fiber.

The following ingredients were added into a confectionery mixing vessel; 10# Milk Confectionery Coating and it was melted and brought to 125° F. To this was then added 21.2% of the total coating weight of the fat emulsion prepared in accordance with Example 5, used for confectionery coatings and Canola oil at a 2 to 1 rate. The amount added was 1.4 oz. fat emulsion of Example 5 and 0.7 oz. Canola oil. About 1 gram of Soy Lecithin then was added as an emulsifier. The content was then mixed for several minutes until it became homogenous and completely emulsified into a uniform structure. Then additional fat emulsion from Example 5 was added to the contents at 1 oz. along with 1.4 g Soy Lecithin and the contents were mixed for several minutes until homogeneous. A final 1 oz. fat emulsion of Example 5 was added to the contents at 1 oz. along with 1.4 g Soy Lecithin and the contents were mixed for several minutes until homogeneous. Then 0.001% of total mixture or 0.4 g of Tocopherols was added to the mixture to maintain freshness. The mixture continued to mix in the heated confectionery mixing vessel at 125° F. for several hours to create a smooth creamy intense mouth feel finished Milk Confectionery Coating with 30% less saturated fat and 30% less sugar as well as less total fat, calories and dietary fiber.

### Example 7

The example provides for a yogurt compound confectionery coating with improved nutritional structure that includes lower saturated fat, lower sugar, lower total fat and beneficial dietary fiber.

The following ingredients were added into a heated confectionery mixing vessel; 10# Yogurt Compound Confectionery Coating and melted until it reached 125° F. To this then was added 21.2% of the coating weight of the fat emulsion from Example 5 used for confectionery coatings and Canola oil at a 2 to 1 rate. This amounted to 1.4 oz. fat emulsion from Example 5 and 0.7 oz. Canola oil. Then about 1 gram of Soy Lecithin was added as an emulsifier. The contents were then mixed for several minutes until the mixture became homogenous and completely emulsified into a uniform structure. Then additional fat emulsion of Example 5 was added to the contents at 1 oz. along with 1.4 g Soy Lecithin and the contents were mixed for several minutes until homogeneous. A final 1 oz. fat emulsion of Example 5 was added to the contents at 1 oz. along with 1.4 g Soy Lecithin and the contents were mixed for several minutes until homogeneous. Then 0.001% of total mixture or 0.4 g of Tocopherols were added to the mixture to maintain freshness. The mixture continued to mix in the heated confectionery mixing vessel at 125° F. for several hours to create a smooth creamy intense mouth feel finished Yogurt Confectionery Coating with 30% less saturated fat and 30% less sugar as well as less total fat, calories and dietary fiber.

### Example 8

The example provides for a Chocolate Confection with Improved Nutritional Structure that includes lower saturated fat, lower sugar, lower fat, lower calories, and dietary fiber.

The following ingredients were added into a chocolate melting and tempering machine; 10# semi-sweet chocolate into a heated confectionery tempering and mixing vessel and melted until it reaches 110° F. To this then was added 20% of the coating weight of the fat emulsion from Example 4, and Canola oil at a 2 to 1 rate. This amounted to 1.35 oz. fat emulsion from Example 4 and 0.65 oz. Canola oil. Then about 1 gram of Soy Lecithin was added as an emulsifier. The contents were then mixed for several minutes until the mixture became homogeneous and completely emulsified into a uniform structure. Then additional fat emulsion of Example 4 was added to the contents at 1 oz. along with 1.4 g Soy Lecithin and the contents were mixed for several minutes until homogeneous. A final 0.8 oz. fat emulsion of Example 4 was added to the contents with 1.2 g Soy Lecithin and the contents were mixed for several minutes until homogeneous. Then 0.001% of total mixture or 0.38 g of Tocopherols was added to the mixture to maintain freshness. The mixture continued to mix in the heated confectionery mixing vessel until homogenous for several hours. Then the temperature was slowly lowered to 88° F. and the chocolate tempered using the chocolate tempering and mixing vessel machine. The finished Chocolate Confection had a rich and creamy mouth feel and 25% less saturated fat and sugar when compared to average semi-sweet chocolate and has less total fat and calories, and additional dietary fiber.

The above three examples of a chocolate coating, a yogurt compound coating, and a chocolate confection all have improved nutritional contents and excellent taste, texture and mouth feel similar to full saturated fat content products. These examples are not limiting and those skilled in the art of producing chocolate coatings and chocolate would understand that a wide range of additional chocolate coatings, compound coatings or chocolate confections could be made within the present invention.

The above eight examples provide compositions that can produce a varied result in the final food product to which they are added depending on the desired rheological characteristics necessary in the food. The preferred embodiments described herein can create a wide range of different viscosity fat emulsion compositions based on varying of certain ingredients within the invention that can provide positive health benefits, when compared to the negative health benefits of high sugar and unhealthy fats that the fat emulsion compositions can replace within a wide range of foods.

### Example 9

This example provides a taste masking composition for docusate sodium that can be formed into a powder that can be mixed with a liquid and ingested.

The taste masking composition had the following ingredients, in the amount shown below:

a. an edible oil component at 4.52% (Cocoa butter);

b. a vegetable glycerin component at 4.15%;

c. fiber component at 32.3% (Inulin);

d. a corn starch component at 34.4%;

e. a glycerin and water based liquid strawberry extract component at 6%;

f. a sunflower oil based liquid lemon extract component at 4.3%;

g. a water and alcohol based vanilla extract component at 3.2%;

i. a sugar alcohol sweetener component at 4.8% (Xylitol 3.8% and Sucralose at 1%);

h. a organic acid component at 4.23% (Ascorbic acid 3.13% and Citric Acid 1.11%):

i. a soy lecithin emulsifier component at 1.04%;

j. a 50% docusate sodium polyethylene glycol 400NF suspension at 1.06%;

The composition was prepared by first adding the cocoa butter, glycerin, strawberry extract, lemon extract, and vanilla extract to a mixing and heating vessel, and heating them to 120° F. The inulin component and corn starch components then were added as the temperature was raised to 140° F. and continuously mixed so that the inulin and corn starch ingredients completely wetted out and the composition was clump free. The Xylitol, sucralose, ascorbic acid, and citric acid then were added to the composition, while mixing, and the temperature was raised to 150° F. making sure that the sugar alcohols and organic acid crystals dissolved completely. Once the sugar alcohol and organic acid crystals dissolved after several minutes of mixing under the 150° F. temperature, the 50% docusate sodium polyethylene glycol 400NF suspension was added to the fat emulsion and the composition was mixed under shear for 20 minutes. At the end of the 20 minutes of mixing the composition became extremely viscous and began to harden as it was removed from the heat.

The composition was allowed to cool to room temperature and then was chopped into small pieces and an anti-caking agent like calcium carbonate or silicone dioxide was added to the pieces as well as additional sugar alcohol sweetener in the form of Xylitol. This group of ingredients was then milled with a kitchen grade grinding machine. The subsequent powder created was a flowable powder with excellent color and consistency. This subsequent powder contained 50 mg of docusate sodium per 10 grams of finished weight material. This level of docusate sodium allowed the powder to deliver therapeutic levels of docusate sodium in a mixable solvent like water, juice, milk, and the like. Tests of this specific docusate sodium flowable powder showed that 30 grams of the powder or 150 mg of docusate sodium can be dissolved and suspended in 6 oz. of solvent. This permitted the docusate sodium-based powder to deliver dosing for children all the way to adults. A typical children's recommended dose of docusate sodium is 150 mg per day, and an adult is 300 mg per day.

Large scale powder processing methods could be used for creating a consumer docusate sodium powder delivery method product. The industry has well established methods for producing flowable powders and the present invention includes these methods of milling. The final product could be marketed in sachets or individual packets or as a bulk container with a suggested serving size using a scoop or measurable suggested dose.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An active ingredient-containing fat emulsion comprising: (a) from about 0.3% to about 40% by weight of the composition of an edible oil; (b) from about 0.001% to about 70% by weight of the composition of a dietary fiber component; (c) from about 1% to about 60% by weight of the composition of a water component or glycerin component; (d) from about 0.001% to about 70% by weight of the composition of a thickening component; and (e) from about 0.001% to about 50% by weight of the composition of an active ingredient.

2. The fat emulsion as claimed in claim 1, further comprising from about 0.003% to about 40% of flavorant component.

3. The fat emulsion of claim 2, wherein the flavorant component is selected from the group consisting of lemon extract, strawberry extract, vanilla extract, and mixtures and combinations thereof.

4. The fat emulsion of claim 1, further from about 0.001% to about 25% of a sugar alcohol component.

5. The fat emulsion of claim 1, further from about 0.001% to about 15% of an organic acid component.

6. The fat emulsion of claim 1, further comprising one or more additives selected from the group consisting of a flavor component, a color component, a preservative component, a stabilizing component, gelatin, gellan gum, xanthan gum, pectin, carrageenan, cellulose gum, gum arabic, modified starch, citric acid, malic acid, lactic acid, fumaric acid, tartaric acid and adipic acid, a nutraceutical component, a vitamin, a mineral, an additional medicine component, or combinations thereof.

7. The fat emulsion of claim 1, wherein the edible oil component is present in an amount of from about 4% to about 15%, based on the total weight of the composition, and wherein the edible oil component is selected from the group consisting of cocoa butter, canola oil, soybean oil, safflower oil, sunflower oil, sesame oil, walnut oil, olive oil, flaxseed oil, chia seed oil, almond oil, corn oil, grape seed oil, peanut oil, coconut oil, and combinations thereof.

8. The fat emulsion of claim 1, wherein the dietary fiber component is present in an amount of from about 10% to about 40%, by weight of the composition, and wherein the fiber component is selected from one or more of the group consisting of naturally derived soluble fiber, naturally derived inulin, inulin extract, synthetic inulin, fructooligosaccharides, galacto-oligosaccharides, xylooligosaccharides, oligo derivatives of starch, husks, brans, psyllium, polysaccharides, starches; polycarbophil, lignin, arabinogalactans, chitosans, oat fiber, soluble corn fiber, non-digestible corn dextrin, non-digestible wheat dextrin, locust bean gum and derivatives thereof, hydroxypropylmethyl cellulose (HPMC), pectin, and mixtures thereof.

9. The fat emulsion of claim 1, wherein the composition comprises from about 4% to about 10% by weight glycerin.

10. The fat emulsion of claim 1, wherein the composition comprises from about 10% to about 35% by weight of corn starch as the thickening component.

11. The fat emulsion of claim 1, wherein the active ingredient is selected from a pharmaceutical ingredient, a nutraceutical ingredient, an over the counter medicin, a prescription medicine, a wellness ingredient, and mixtures thereof.

12. The fat emulsion of claim 11, wherein the active ingredient is docusate sodium.

13. A fat emulsion comprising the following components, each by weight of the total composition:
(a) from about 4% to about 15% of a cocoa butter component;
(b) from about 10% to about 40% of an inulin dietary fiber component;
(c) from about 4% to about 10% of a vegetable glycerin component;
(d) from about 10% to about 25% of a corn starch thickening component;
(e) from about 5% to about 8% of a glycerin and water based strawberry extract component;
(f) from about 4% to about 8% of a sunflower oil based lemon extract component;
(g) from about 3.5% to about 8% of a water and alcohol based liquid vanilla extract component;
(h) from about 3% to about 8% of a sugar alcohol sweetener component with xylitol and sucralose;
(i) from about of 1% to about 3% of a liquid soy lecithin emulsifier component; and
(j) from about 1% to about 20% a docusate sodium polyethylene glycol 400NF suspension active ingredient component.

14. A fat emulsion comprising the following components, each by weight of the total composition:
(a) about 4.5% of a cocoa butter;
(b) about 32.3% of inulin;
(c) about 4.2% of vegetable glycerin;
(d) about 34.4% of corn starch;
(e) about 6% of a glycerin and water based strawberry extract;
(f) about 4.3% of a sunflower oil based lemon extract;
(g) about 3.2% of a water and alcohol based liquid vanilla extract;
(h) about 4.8% of a sugar alcohol sweetener comprising about 3.8% xylitol and about 1.0% sucralose;
(i) about of 1% of a liquid soy lecithin emulsifier; and
(j) about 1% of a 50% docusate sodium polyethylene glycol 400NF suspension.

15. A method of making a homogeneous fat emulsion comprising:
(a) mixing from about 0.3% to about 40% by weight of an edible oil, from about 1% to about 40% by weight of water or glycerin, and from about 0.003% to about 40% by weight of a flavorant in a suitable mixing vessel capable of mixing and heating;
(b) mixing the components and heating the mixing vessel to a temperature within the range of from about 100° F. to about 150° F.;
(c) adding from about 0.001% to about 70% of a dietary fiber, and heating the mixing vessel to a temperature within the range of from about 120° F. to about 160° F.;
(d) adding from about 0.001% to about 70% of a thickening component and mixing for a period of time sufficient to adequately mix and dissolve the thickening component, while maintaining the temperature to within a range of from about 120° F. to about 160° F.;
(e) optionally cooling;
(f) adding from about 0.001% to about 50% of an active ingredient; and
(g) formulating the mixture into a form suitable for delivery to a subject.

16. The method as claimed in claim 15, further comprising adding from about 0.001% to about 25% of a sugar alcohol component, from about 0.001% to about 15% of an organic acid component, and from about 0.001% to about 10% of an emulsifier component prior to process (g), and raising the temperature of the mixture to a temperature within the range of from about 140° F. to about 170° F.

17. The method as claimed in 15, wherein, after process (f), the mixture is mixed under shear for a period of time of from about 5 to more than 30 minutes until the mixture thickens into a homogeneous fat emulsion.

18. The method as claims in claim 17, wherein formulating the mixture comprises grinding the mixture to form a powder.

19. The method of claim 15, wherein the active ingredient is selected from a pharmaceutical ingredient, a nutraceutical ingredient, an over the counter medicin, a prescription medicine, a wellness ingredient, and mixtures thereof.

20. The method of claim 19, wherein the active ingredient is docusate sodium.
